(19) United States
(12) Patent Application Publication
(10) Pub. No.: US 2024/0389926 A1
(43) Pub. Date: Nov. 28, 2024

(54) TRANSDERMAL ALLERGY TESTING

(71) Applicants: DANMARKS TEKNISKE UNIVERSITET, Kongens Lyngby (DK); KØBENHAVNS UNIVERSITET, Kobenhavn K (DK); HERLEV GENTOFTE HOSPITAL, Hellerup (DK)

(72) Inventors: Stephan Sylvest KELLER, Kongens Lyngby (DK); Claudia CAVIGLIA, Kongens Lyngby (DK); Bettina Margrethe JENSEN, Hellerup (DK); Lars Kærgaard POULSEN, Hellerup (DK)

(21) Appl. No.: 18/694,477

(22) PCT Filed: Sep. 22, 2022
(86) PCT No.: PCT/EP2022/076408

§ 371 Date: Mar. 22, 2024

(30) Foreign Application Priority Data
Sep. 22, 2021 (EP) 21198279.8

(51) Int. Cl. A61B 5/00; A61B 5/145; A61B 5/1473

(52) U.S. Cl. CPC A61B 5/411; A61B 5/14546; A61B 5/1473; A61B 5/443; A61B 5/685

(57) ABSTRACT

Allergy is one of the world's most common chronic conditions. The state of the art method for allergy testing is the skin prick test (SPT) where different allergens are introduced into the skin of the tested person. This method is at best semi-quantitative, a relatively low number of tests can be performed and patients might experience unpleasant reactions. This invention is a novel allergy test providing fast, accurate and quantitative monitoring of allergic reactions in the skin. As a novelty, microneedle-based electrochemical sensor arrays are used to introduce allergens in the skin and perform electrochemical detection of biomarkers released from activated mast cells in the interstitial fluid.

### TECHNICAL FIELD OF THE INVENTION

The present invention relates to a system for detecting an allergic reaction in the skin. In particular, the present invention relates to a system that quantitatively monitors an allergic reaction in the skin.

### BACKGROUND OF THE INVENTION

Allergy is one of the world's most common chronic conditions and caused by immunoreaction of the human body towards in principle harmless substances called allergens. An increasing number of people is diagnosed with allergy towards insect stings, pollen, dust, animal dander, food or drugs.

In 2013, the World Allergy Organization (WAO) reported that an estimated 240-550 million people around the globe are affected by food allergy, 300 million suffer from asthma and 400 million from allergic rhinitis (hay fever). Evidence based on epidemiological studies show that the prevalence of allergic diseases is rising all across the world. Epidemiological studies in Europe show that chronic allergic diseases affect more than 150 million people on this continent. The European Academy of Allergy and Clinical Immunology (EAACI) considers allergy as a public health concern of growing proportions, as it predicts that with the current trend more than half of the European population will develop at least one type of allergy by 2025.

Allergy has indeed a major negative impact on the patients' life quality including physical and mental well-being, social relations, educational and career progression and productivity. Since the onset of the most allergic diseases is during the early years of life, children and youth suffer most from the impaired life quality. In Europe, more than 100 million days of absence from school and work due to asthma and hay fever are recorded every year. The economic burden of the allergic diseases on the national health care systems in EU is estimated to be between 55-151 billion Euros per year. In the United States, more than 24 billion US Dollars are spent annually on tackling food allergy in children alone. According to EAACI, 45% of the allergy cases in Europe are not properly diagnosed. A study among US adults shows that 1 out of 5 American adults believe that they suffer from food allergy, whereas only 1 out of 20 refer to physicians to get a proper clinical diagnosis. Early accurate diagnosis of allergy and the causative allergen(s) is crucial for receiving exposure prevention guidelines and an effective treatment. Furthermore, getting a diagnosis as close as possible to the onset of the allergic disease is vitally important for avoiding further complications, improving the patients' quality of life and for cutting down the expenditure on medications and acute medical services.

The state of the art method for allergy testing is the skin prick test (SPT) where different allergens are introduced into the skin of the tested person.

The SPT has several limitations mainly related to the fact that visible changes of the skin have to be assessed. The evaluation of the SPT requires an experienced health care professional and can to a certain degree be subjective. Furthermore, the analysis is non quantitative in the sense that mainly a yes or no answer can be obtained for a given allergen, which does only allow an indicative assessment of the severity of an eventual allergy. There is a certain risk for false negatives due to absence of visible reactions on the skin because of a different or too low response (e.g. itching only) or false positives due to immunoreactions induced by the SPT itself (e.g. skin redness). In terms of patient compliance, the SPT is uncomfortable for the test person. It is performed on the forearms of the patient, which typically allows to include a maximum of around 10 allergens in a test sequence. Finally, it takes approximately 15-20 min until the inflammatory reaction becomes visible and including manual assessment the total test duration of up to 1 hour is in particular a challenge for diagnosis of allergy with children.

Alternatively, blood tests such as the histamine release test (HRT) and the basophil activation test (BAT) have been established. These methods allow a more quantitative assessment of the allergic reaction. However, they require sample pre-treatment, expensive equipment in specialized laboratories and a short time between blood sampling and measurements to prevent sample degradation.

US patent application 2008/0154149 (to Bayer Healthcare LLC) discloses a method of using a diffusion-based, continuous-monitoring system to analyze the effect of an allergen on skin includes creating and maintaining a diffusion channel in an area of skin. The skin is contacted with an allergen. Information related to at least one antibody, histamine and/or leukotriene is continuously monitored for a desired duration via a diffusion-based, continuous-monitoring device. Information related to the at least one antibody, histamine and/or leukotriene is analyzed to determine the effect of the allergen on the skin. It is briefly mentioned that an electrochemical analysis may be performed in this continuous-monitoring system, but any technical details of such an electrochemical system for allergy testing are completely absent.

Hence, an improved method for allergy testing would be advantageous, and in particular a more efficient and/or reliable allergy test, solving the above mentioned issues and providing a quantitative assessment of allergy would be advantageous.

### SUMMARY OF THE INVENTION

Thus, an object of the present invention relates to an allergy test providing quantitative monitoring of allergic reactions in the skin.

In particular, it is an object of the present invention to provide an allergy test that solves the above mentioned problems of the prior art by being more accurate, quantitative and fast compared to the SPT.

Thus, one aspect of the invention relates to a system for determining an allergic reaction in skin, the system comprises:
allergen or allergens,
a microneedle array with a microneedle configured to receive said allergen or allergens, for introducing said allergen or allergens into the skin, wherein the microneedle is able to detect an electrochemical signal as a response to the introduced allergen, and
a monitoring device for receiving the electrochemical signal detected by said microneedle, said electrochemical signal being indicative for an allergic reaction in the skin.

Another aspect of the present invention relates to a microneedle array comprising:
a microneedle configured to receive an allergen, wherein the microneedle comprises an electrochemical sensor configured to receive, process and/or record an electrochemical signal triggered by the allergen.

Yet another aspect of the present invention is to provide a set of parts for detecting an allergic reaction in the skin, the set of parts comprising:
allergen or allergens,
a microneedle array according to the invention
at least one monitoring device for receiving the electrochemical signal detected by said microneedle array in the skin, said electrochemical signal being indicative for an allergic reaction in the skin.

### BRIEF DESCRIPTION OF THE FIGURES

FIG. 1 shows a non-limiting example of the microneedle-based sensor array for electrochemical sensing. A) microneedle array coated with different allergens (marked by light and dark gray). B) A cross-sectional view of the microneedle-based electrochemical sensor when applied to the skin C) (1) Allergen release from microneedles, (2) mast cell activation and (3) degranulation, and (4) electrochemical sensing of biomarkers at the distal end of the microneedles.

FIG. 2 shows a non-limiting example of three different sensor configurations. A1-4) Configuration 1—Direct electrochemical sensing in the skin; (A1) microneedle sensor with allergens (AL); (A2) release of AL in the skin tissue and activation of mast cells (MC); (A3) release of biomarkers (BM) from MC; (A4) oxidation of BM at microneedle electrode. (B1-4) Configuration 2—Electrochemical enzymatic biosensing in the skin; (B1) microneedle sensor with enzymes (EN) and allergens (AL); (B2) release of AL in the skin tissue and activation of mast cells (MC); (B3) release of biomarkers (BM) from MC; (B4) oxidation of BM by EN on microneedle electrode and electrochemical sensing. (C1-4) Configuration 3—Electrochemical ELISA-based immunosensing outside the skin; (C1) microneedle sensor with capture antibody (cAb) and allergens (AL); (C2) release of AL in the skin tissue and activation of mast cells (MC); (C3) release of biomarkers (BM) from MC and binding to cAb; (C4) removal of microneedles from the skin, introduction of detection antibody (dAb) labelled with an electroactive label (EL) and electrochemical quantification of EL

FIG. 3 shows open circuit potentiometry measurements of blank samples (baselines) followed by histamine measurements in mast cell supernatant for two different cell densities corresponding to two different histamine concentrations.

FIG. 4 shows a non-limiting example of the microneedle-based electrochemical sensor design and fabrication. (A) Illustration of the two main designs of microneedle-based electrochemical sensors: (A1) in plane microneedles and (A2) out of plane microneedles; (B) Fabrication process of Si-based in-plane microneedles with integrated pyrolytic carbon microelectrodes (detailed description see text); (C) SEM images of Si-based in plane microneedles with pyrolytic carbon electrodes after completed fabrication; (D) Fabrication process of out of plane pyrolytic carbon microneedle: (D1) 3D design of interior and exterior structure; (D2) 3D printed polymer microneedle; (D3) Carbon microneedle after pyrolysis.

FIG. 5 shows (A) Schematic design of Si chip with array of in-plane Si microneedles (IPSMN) and SEM images of the IPSMN with five different tip designs for penetration testing. L=500 μm, w=400 μm, t=180 μm. (B) Penetration forces measured in hydrogel-based in vitro skin tissue model for IPSMN with L=500 μm, w=400 μm and variable thickness (180 μm, 350 μm and 500 μm).

FIG. 6 shows (A) Histology cross-section of a penetration hole caused by a triangle shaped microneedle (L=500 μm, W=400 μm); (B) Histology cross-section of a penetration hole caused by a pencil shaped microneedle (L=1000 μm, W=400 μm); (C) Quantitative analysis of the hole depth induced by different IPSMN in comparison with the skin prick test needle.

FIG. 7 shows three examples of microneedle electrodes serving as transducer in the electrochemical sensor; (A) solid microneedle electrodes; (B) coated microneedle electrodes; (C) hollow microneedle electrodes

FIG. 8 shows two possible configurations for electrochemical measurements in the skin: (A) Setup where part of the microneedles served as working electrode (WE) and the others as counter electrode (CE); (B) interdigitated setup where the microneedles alternatively serve as WE and CE.

FIG. 9 shows examples of methods for allergen integration on the microneedles and allergen introduction in the skin: (A) Deposition of allergens on the surface of solid microneedles as coating or film and release of allergens in the skin tissue; (B) Dispensing of allergens in the skin using hollow microneedles; (C) Multiplexing of allergen deposition, where 4 different allergens are deposited on the microneedles by dip coating; (D) Multiplexing of allergen dispensing by capillary filling of microneedles with 4 different allergens

FIG. 10 shows histology images of biopsies of pig neck skin (upper panels) and human breast skin (lower panels) samples with penetration holes after microneedle insertion for 2 seconds, 30 seconds or 60 seconds. The microneedles were coated with Rhodamin B and the presence of Rhodamin B is shown by the black circles.

FIG. 11 shows the results from dermal delivery of compound 48/80 in Brown Norway rats. Rats were treated with the 48/80 compound or PBS either injected intradermally or inserted with coated microneedles and 1% Evan's blue dissolved in PBS was injected in the tail vein of the rats. The animals were sacrified after 15 minutes and the abdominal skin was excised. (A) An image of excised rat skin, stretched to its original size and fixed on Styrofoam using pin needles. The different concentrations and insertion sites of 48/80 are shown on the image (5 μg/mL, 10 μg/mL, 20 μg/mL, 30 μg/mL, 40 μg/mL). In addition, 48/80 was injected intradermally as a positive control along with two negative controls of PBS; one of which was injected intradermally. The black coloration in the area around the needle injection point show the coloration from the Evan's blue. (B) A quantification of the coloration from Evan's blue in figure A) stratified into male and female rat groups.

FIG. 12 shows results from quantifying immunoresponses to birch pollen extract (BPE) and bet v1 birch allergen in naive and sensitized rats. (A) The experimental setup. Teams 1 and 3 are naive rats whereas teams 2 and 4 are sensitized rats. Each group consists of four males and four females. The sensitization in teams 2 and 4 was performed by injecting BPE in PBS by intraperitoneal (IP) injection whereas the naive rats received PBS (negative control). Immunizations occurred once a week for four consecutive weeks (Day 0, 7, 14 and 21). Blood samples were collected from all animals before immunization. After the last IP post-immunization, the rats were subjected to an ear swelling test (EST) with 10 μg BPE in PBS in the left ear and with 3 μg Bet v1 in PBS in the right ear on Day 25 and 27. Rats were sacrificed on days 28 and 29 at which point the microneedle (teams 3 and 4) and intradermal injection (teams 1 and 2) delivery of allergen experiments were conducted. (B) Schematic illustration of the 8 sites of insertion of the different solutions that are inserted via microneedles or intradermal injections. Each site is lettered from A to H: A, B, C are Bet v1 allergen; D, E, F are birch pollen extract (BPE); G is 48/80 (positive control); H is PBS (negative control). Each solutions' insertion site has been randomized i.e. each rat has different insertion places for each solution. (C) Images of rat skin from naive and sensitized rats that have received BPE, bet v1, 48/80 or PBS by intradermal injections or microneedle insertions. Since the insertion site of each solution is randomized, the order of the solutions are different compared with the schematic illustration of FIG. 12B. The black coloration from Evan's blue is visible for some samples. (D) A quantification of the coloration from Evan's blue in figure C) stratified into naive or sensitized rats treated with either BPE or bet v1.

The present invention will now be described in more detail in the following.

### DETAILED DESCRIPTION OF THE INVENTION

### Definitions

Prior to discussing the present invention in further details, the following terms and conventions will first be defined:

### Allergen

The term “allergen” when used herein is defined as a substance that induces allergy, which is an abnormal immune response, wherein the immune system reacts to a substance that would otherwise be harmless to the body. An allergen is capable of stimulating a type-I hypersensitivity reaction through immunoglobulin E (IgE) responses.

### Allergen Immunotherapy

The term “allergen immunotherapy” when used herein is defined as one of the few clinically proven therapeutic vaccination strategies given to patients. By controlled administration of increasing allergen doses with time, allergic reactions are gradually suppressed and the subject can develop tolerance towards the specific allergen.

### Amine Oxidases

The term “amine oxidases” when used herein is defined as a class of enzymes catalyzing the oxidative cleavage of mono-, di-, and polyamines to generate other biologically active molecules such as aldehydes, ammonia, and hydrogen peroxide.

### Biomarker

The term “biomarker” when used herein is defined as a measurable indicator of some biological condition.

### Biorecognition Element

The term “biorecognition element” when used herein is defined as the component in a biosensor (e.g. antibody, enzyme, aptamer) providing analyte specific detection (Morales 2018).

### Biosensing

The term “biosensing” when used herein is defined as a the detection of an analyte (e.g. biomarker or chemical compound) with a device using a biorecognition element and a transducer. The biorecognition element (e.g. antibody, enzyme) allows the specific capture of the analyte of interest, while the transducer (e.g. electrochemical sensor, optical sensor) allows the conversion of the capture event in a measurable physical signal.

### Counter Electrode (CE)

The term “CE” when used herein is defined as the electrode in an electrochemical sensor completing the electrochemical circuit with a working electrode (WE).

### Dermis

The term “dermis” when used herein is defined as a layer of the skin between the epidermis and the subcutaneous tissues.

### Diamine Oxidase (DAO)

The term “DAO” when used herein is defined as a an enzyme involved in the metabolism, oxidation and inactivation of amines such as histamine or other polyamines in plants, animals and the human body.

### Epidermis

The term “epidermis” when used herein is defined as the outermost layer of the skin, found on top of the dermis.

### Mast Cell

The term “mast cell” when used herein is defined as a specific type of resident white blood cells found in the skin tissue. During allergic reactions, antigens bind to IgE antibodies on sensitized mast cells inducing degranulation and release of biomarkers such as histamine, tryptase, cytokines and heparin.

### Microneedle

The term “microneedle” when used herein is defined as a microscopic needle with dimensions in the sub-mm to mm range typically produced by microfabrication methods.

### Minimal Invasive

The term “minimal invasive” when used herein is defined as a method, which does not contain a step representing a substantial physical intervention on the body. Further, the method or intervention does not require highly specialized professional medical expertise to be carried out and does not contain a substantial health risk.

### Potentiostat

The term “potentiostat” when used herein is defined as an electronic equipment used for electroanalytical measurements, managing the application of a voltage and measuring the current flow through the electrochemical system or vice versa.

### Prussian Blue

The term “Prussian Blue” when used herein is defined as a metal hexacyanoferrate frequently used as non-enzymatic electrocatalyst in electrochemical sensing.

### Prussian White

The term “Prussian White” when used herein is defined as the reduced form of Prussian Blue.

### Subject

The term “subject” when used herein is defined as a human or non-human species of mammals including primates, livestock animals (e.g. sheep, cows, pigs, horses, donkey, goats), laboratory test animals (e.g. mice, rats, rabbits, guinea pigs, hamsters) and companion animals (e.g. dogs, cats). The present invention has applicability, therefore, in human medicine as well as having livestock and veterinary and wildlife applications. In a preferred embodiment, the mammal is a human.

### Working Electrode (WE)

The term “WE” when used herein is defined as the electrode in an electrochemical sensor on which the analyte of interest is detected electrochemically.

It should be noted that embodiments and features described in the context of one of the aspects of the present invention also apply to the other aspects of the invention.

All patent and non-patent references cited in the present application, are hereby incorporated by reference in their entirety.

### The Invention Will Now be Described in Further Details in the Following Non-Limiting Examples.

The invention relates to a system that quantitatively can measure the degree of allergic reaction within a subject. By electrochemically detecting a biomarker released by immune cells as a response to a given allergen in very low concentration, too low for visual determination, it is possible to calculate a value for the given response. The electrochemical signal enables to quantify the degree of allergy at a much more detailed level compared to the well-established skin prick test, which is the go-to method today.

Thus, a first aspect of the invention relates to a system for determining an allergic reaction in the skin, the system comprises:
allergen or allergens,
a microneedle array with a microneedle configured to receive said allergen or allergens, for introducing said allergen or allergens into the skin, wherein the microneedle is able to detect an electrochemical signal as a response to the introduced allergen, and
a monitoring device for receiving the electrochemical signal detected by said microneedle, said electrochemical signal being indicative for an allergic reaction in the skin.

In one embodiment of the present invention, the system can quantitatively measure the degree of allergic reaction in the skin.

In another embodiment, the system is adapted for measuring said electrochemical signal as a response to the introduced allergen so as to provide a quantitive indication for said allergic reaction in the skin, preferably on a relative scale.

An example of measurement on a relative scale could be comparing a signal detected in the presence of an allergen with a signal detected without the presence of an allergen.

The detection of the signal could be either in the same subject or in different subjects.

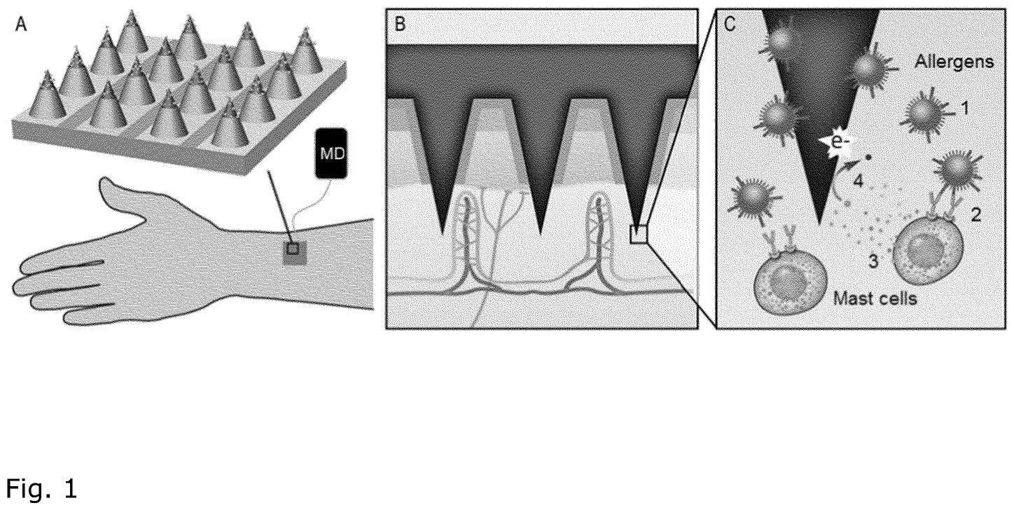

Thus, FIG. 1A+B illustrates an embodiment of a system for determining an allergic reaction in the skin. Said system has a microneedle array (A), which is coupled to a monitoring device (MD). The microneedle array (A) has a microneedle and preferably, a plurality of microneedles, which may be spaced in a linear array, a two-dimensional array or any other spacing desired. The microneedles can be coated with either allergen alone or allergen and enzyme (A), which, when introduced into the skin (B), is able to activate mast cells (C2) present in the interstitial fluid, if the person is allergic towards the introduced allergen (C1). The activated mast cells will release biomarkers (C3), which can be detected by the microneedles and the electrochemical signal will be transferred from the needles to the monitoring device for recording.

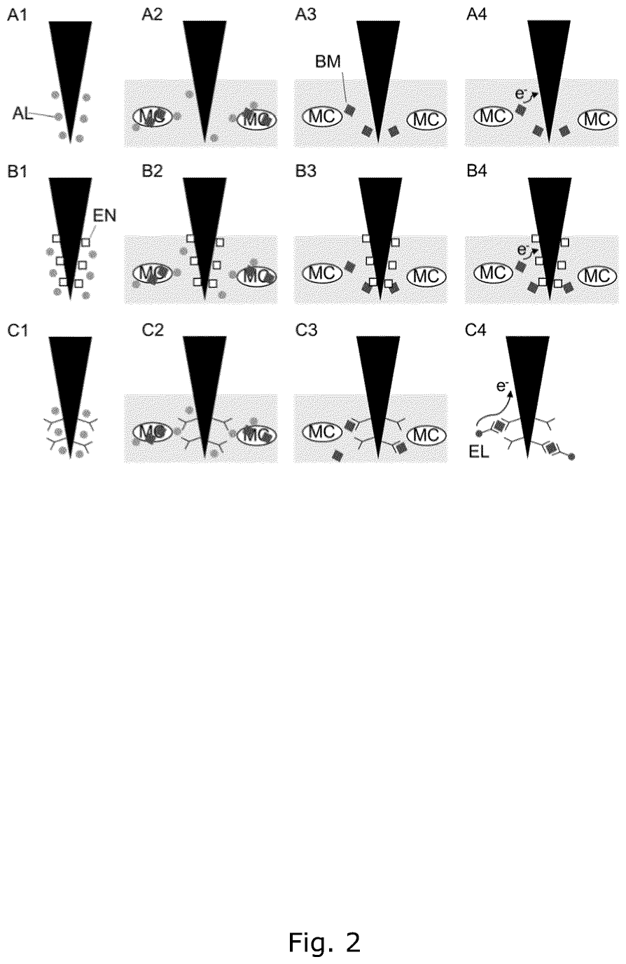

In another embodiment, the microneedles will capture the biomarker and enable detection of the signal outside the skin. This configuration is illustrated in FIG. 2 C1-C4.

In one embodiment, said electrochemical signal is detected by said microneedle in the skin.

FIG. 2 illustrates an embodiment of the system for detecting the electrochemical signal in the skin. FIGS. 2a, 2b and 2c are separate embodiments of the present invention.

As illustrated in FIG. 2a1, allergen (AL) is coated onto the microneedle and when introduced into the skin (illustrated by grey box) the allergen activates mast cells (MC) (FIG. 2a2). The mast cells release biomarker (BM) (FIG. 2a3). The released biomarker is oxidized or reduced at the microneedle-based electrode and the electrochemical signal recorded (FIG. 2a4).

As illustrated in FIG. 2B, an enzyme is used to convert the biomarker, which triggers an electrochemical signal in the microneedle array. Allergen (AL) and enzyme (EN) are coated onto the microneedle (FIG. 2b1), when introduced into the skin (illustrated by grey box) the allergen activates mast cells (MC) (FIG. 2b2). The mast cells release biomarker (BM) (FIG. 2b3). The released biomarker is converted by the enzyme and the product thereafter detected by the needles as an electrochemical signal (FIG. 2b4).

Thus, in one embodiment the microneedle or microneedles according to the invention are configured to receive an enzyme.

As illustrated in FIG. 2C, a biorecognition element is used to catch the biomarker released by the mast cells. The biorecognition element (illustrated as a Y) is immobilized onto the microneedle and allergen is coated on top (FIG. 2c1), when introduced into the skin (illustrated by grey box) the allergen activates mast cells (MC) (FIG. 2c2). The mast cells release biomarker (BM) (FIG. 2c3). The released biomarker are bound by the antibody on the needles (FIG. 2c4), the needles are removed and a secondary antibody to the biomarker labelled with an electroactive label is introduced on the microneedles. The presence of the electroactive label is detected electrochemically by the microneedle electrodes.

Thus, in an embodiment, the enzyme is an enzyme that can convert the biomarker into a product suitable for electrochemical detection.

In another embodiment, the enzyme is an amine oxidase.

In a preferred embodiment, the enzyme coated on the microneedles is histamine oxidase.

In a more preferred embodiment, the enzyme coated on the microneedles is DAO.

In another embodiment, said electrochemical signal is detected by said microneedle outside the skin.

In a further embodiment, the electrochemical signal is detected by said microneedles outside the skin by electrochemical ELISA-based immunosensing.

### Biomarkers

Human mast cells resident in tissue are known to play a key role in allergic inflammation. These granulocytes bind allergen-specific IgE antibodies and become sensitive towards the allergen. The secretory granules of mast cells contain preformed inflammatory mediators such as histamine, heparin, serotonin, tryptase, chymase, a number of other cytokines and growth factors. Next time the individual is exposed to the allergen, activation of the mast cells initiates degranulation which results in the instantaneous release of the inflammatory mediators. The mast cell density in the upper dermis is around 56-64 cells/mm². Histamine can cause inflammation and effects on the vasculature in form of vasodilation which in the worst case can result in anaphylactic shock. In the SPT the mast cells present in the skin are activated and the release of histamine and other biomarkers triggers an immunoreaction which induces local inflammation (wheal and flare reaction).

In one embodiment, the electrochemical signals is triggered by biomarkers released by activated mast cells, preferably the electrochemical signal is triggered by biomarkers selected from the list consisting of histamine, chymase, and tryptase, more preferably the electrochemical signal is triggered by histamine released by activated mast cells.

In another embodiment, the microneedle array can detect signals triggered by said biomarkers in concentration below 900 nM, 800 nM, such as below 400 nM, preferably concentrations below 100 nM.

### The Monitoring Device

The monitoring device used according to this invention is able to receive, process and/or record an electrochemical signal from the electrodes in the microneedles. The recorded signal is stored for later analysis.

Thus, in one embodiment, the monitoring device is configured to receive, process and/or record an electrochemical signal from one, or more, electrode(s) arranged in said microneedle, preferably said electrode(s) are arranged at the distal end of said microneedle.

In another embodiment, the monitoring device is or may comprise a potentiostat.

In a further embodiment, the monitoring device may comprise a potentiostat.

In yet another embodiment, the monitoring device is a potentiostat.

In a further embodiment, the monitoring device is implemented by means of hardware, software, firmware or any combination of these. The monitoring device or some of the features thereof can also be implemented as software running on one or more data processors and/or digital signal processors.

Preferably, the monitoring device may be physically integrated on the microneedle array, either completely with processing of the electrochemical signal(s) on the rear or back side of the microneedle array and readout of the various electrochemical signals, if the size of the array allows.

Alternatively the monitoring device may be structurally and/or functionally integrated to other physical entities with transmission by appropriate wires or wirelessly (e.g. via Bluetoth or other EM signals) of a representation of the electrochemical signal(s) to one or more other entities, for example a computer, a portable computer, a portable tablet, a smart phone etc., where a professional health person may read the electrochemical signals, or parameters derived therefrom, and apply them for analysing and interpreting an allergic condition or state of the subject being tested by the microneedle array.

Thus, the individual elements of the monitoring device may be physically, functionally and logically implemented in any suitable way such as in a single unit, in a plurality of units or as part of separate functional units. The monitoring device forming part of the invention may be implemented in a single unit, or be both physically and functionally distributed between different units and processors.

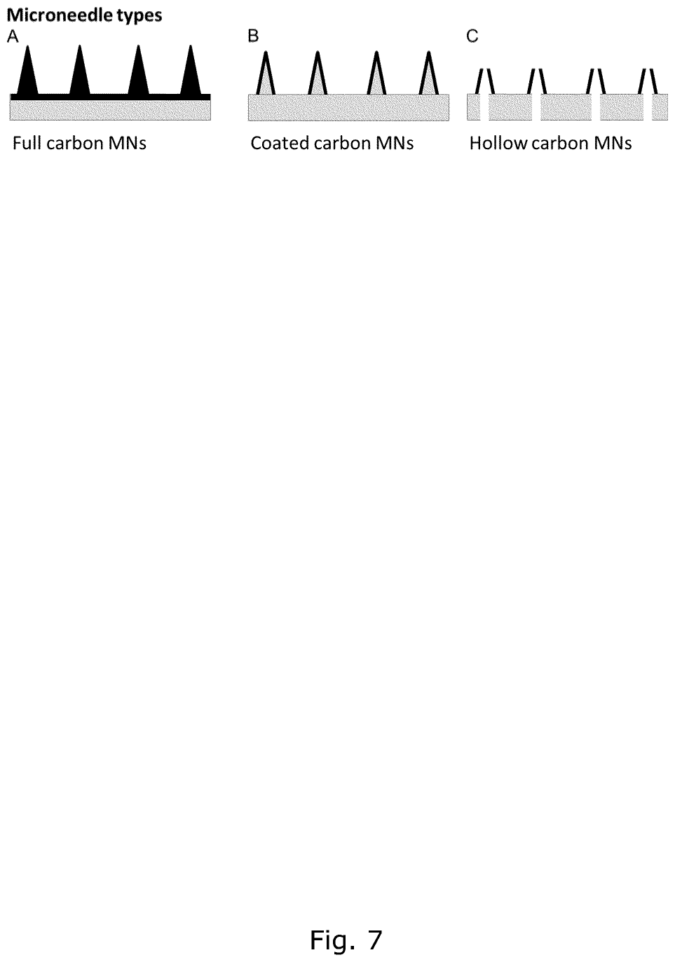

The electrode can be positioned within the microneedle in different ways. The electrode can be the entire microneedle, it can be coated on top of the needle or the electrode can be located at the distal end of the microneedle (FIG. 7).

Thus, in an embodiment, the electrodes are the entire microneedles.

In another embodiment, the electrode is a layer at the surface of the microneedles (coating).

In another embodiment, the electrode can be a wire on and/or in the microneedles.

In yet a another embodiment, the microneedle is a hollow microneedle.

In another embodiment, the electrode is located at the distal end of the microneedle.

### Detection Time

The time used for detection is defined by the time that the allergen requires to initiate the mast cell degranulation combined with the time required for biomarker detection. Mast cell degranulations is instantaneous upon contact with the allergen, while electrochemical detection of biomarkers typically is possible within a few minutes.

Thus, in one embodiment, the allergic reaction can be detected within an hour, such as 30 minutes, such as 20 minutes, such as 10 minutes, such as 5 minutes preferably the allergic reaction can be detected within 20 min, more preferably 10 min.

In another embodiment, the allergic reaction can be detected within 10 minutes.

### The Microneedle Array

An aspect of the present invention relates to a microneedle array comprising: a microneedle configured to receive an allergen, wherein the microneedle comprises an electrochemical sensor configured to receive, process and/or record an electrochemical signal triggered by the allergen.

In the setup as described in FIG. 2b, for the microneedles to be able to detect the electrochemical signal, the biomarker released by the mast cells has to be enzymatically converted.

Thus, in an embodiment, the microneedle is configured to receive both an enzyme and an allergen.

In a preferred embodiment, the enzyme is an amine oxidase.

In another preferred embodiment, the enzyme coated on the microneedles is histamine oxidase.

In a more preferred embodiment, the enzyme coated on the microneedles is DAO.

The microneedle array can be composed of different numbers of microneedles.

In one embodiment, the microneedle array comprises one microneedle.

In another embodiment, the microneedle array comprises a plurality of microneedles arranged in a predetermined pattern.

In a further embodiment, the microneedle array comprises at least 2 microneedles, such as 5 microneedles, such as 10, such as 20, such as 30, such as 50 microneedles, preferably between 10-30 microneedles, more preferably between 15-25, even more preferably the microneedle array comprises 20 microneedles.

In a preferred embodiment, the microneedle array comprises 10-30 microneedles, preferably 15-25 microneedles.

It should be understood that any number of microneedles might be present on the microneedle array. It should be understood that when a plurality of microneedles is present in the middleneedle array, the plurality of microneedles may be identical, almost or substantially identical, or alternatively the plurality of microneedles may be different from each other, both with respect to structure and/or functional way of working.

In order to detect an electrochemical signal and transfer said signal to the monitoring device, the microneedle is connectable to the monitoring device according to the invention.

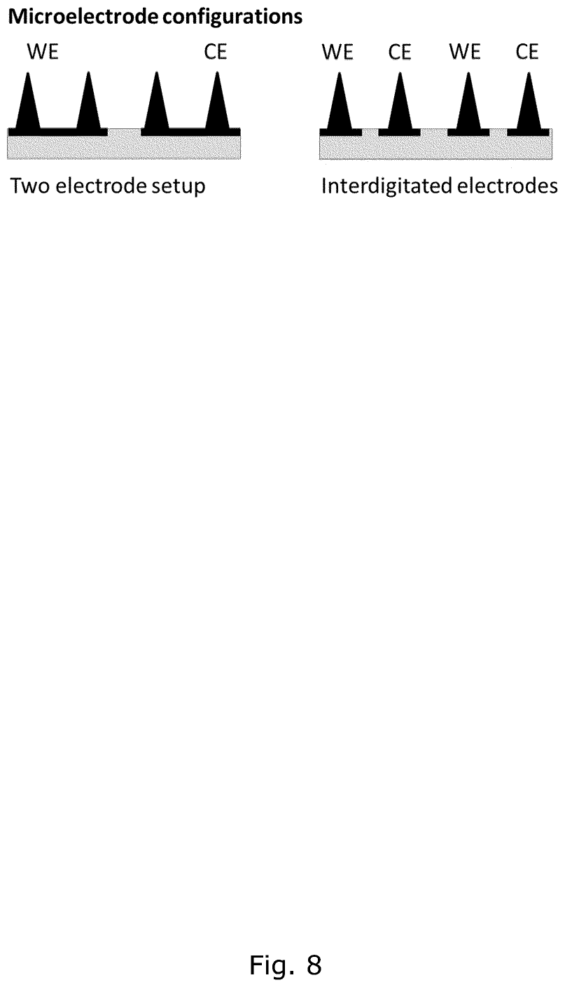

As seen in FIG. 8, the microneedle can be connected either individually or in groups of 2 or more to the monitoring device.

In one embodiment, the microneedles are connectable to a monitoring device in groups 2 or more, such as 3 or more, such as 4 or more, such as 5 or more, such as 10 or more, such as 20 or more.

In another embodiment, the microneedles are individually connectable to a monitoring device.

The electrochemical sensor comprises two different electrodes in order to be able to perform electrochemical sensing. Thus, the electrochemical sensor comprises a working electrode (WE), which measures the biomarker and a counter electrode (CE) which complete the electrical circuit.

The electrochemical sensor may for each WE have a corresponding CE or the electrochemical sensor may have a global CE, which is the same for all or for a group of WEs in the sensor.

Thus, in one embodiment of the present invention, each WE has a corresponding CE.

In another embodiment, one CE is present for a group of WEs.

The CE and WE can be present on separate microneedles or be integrated in the same microneedle.

Thus, in one embodiment, each needle comprises either a WE or a CE.

In another embodiment, each needle comprises both a WE and a CE.

The material used to fabricate the microneedles or at least part of the microneedles, has to allow electrochemical sensing and signal transduction.

Thus, in one embodiment, the microneedle array is made of a material configured to detect an electrochemical signal according to the invention.

In another embodiment, only part of the microneedle array is made of a material configured to detect an electrochemical signal according to the invention.

In another embodiment, the microneedle array comprises a conductive material, preferably the microneedle array comprises pyrolytic carbon.

In another embodiment, parts of the microneedle array comprise a conductive material, preferably parts of the microneedle array comprise pyrolytic carbon.

In yet another embodiment, the microneedle array comprises conductive material and a dielectric material.

In yet a further embodiment, the dielectric material is silicon dioxide, silicon nitride, fused silica, ceramics, glass or a polymer.

FIG. 7 shows an embodiment of the microneedles in the microneedle array. As seen in FIG. 7A the needles can be entirely of pyrolytic carbon, or as seen in FIG. 7B coated with a layer of pyrolytic carbon. FIG. 7C shows an embodiment of the invention where the needles are made of hollow pyrolytic carbon.

Thus, in one embodiment, the microneedle can be made entirely of pyrolytic carbon.

In another embodiment, the microneedle can be coated with pyrolytic carbon.

In a further embodiment, the microneedle can be partially coated with pyrolytic carbon.

In yet another embodiment, the microneedle can be hollow pyrolytic carbon.

Further, the dimensions of the microneedle or microneedles used in the microneedle array can vary in a number of ways.

It has to be understood that the microneedle can be of any shape. Further, the skilled person in microneedle design will readily understand that various shapes and structures of microneedles, various types of microneedles, or various functions of microneedles may be applied and implemented in the context of the present invention, once the teaching and principle of the present invention is fully understood.

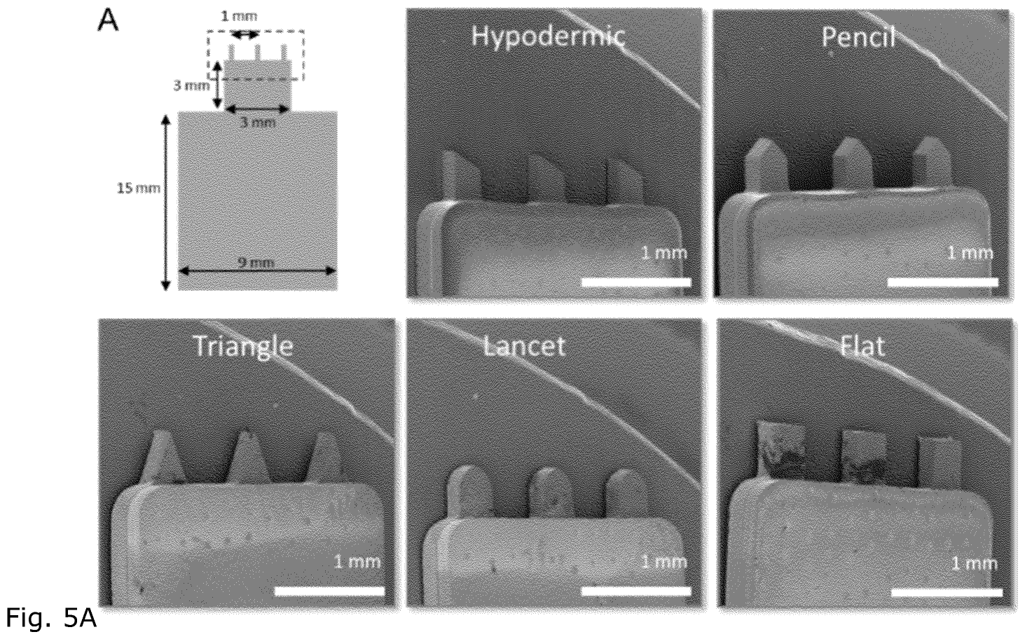

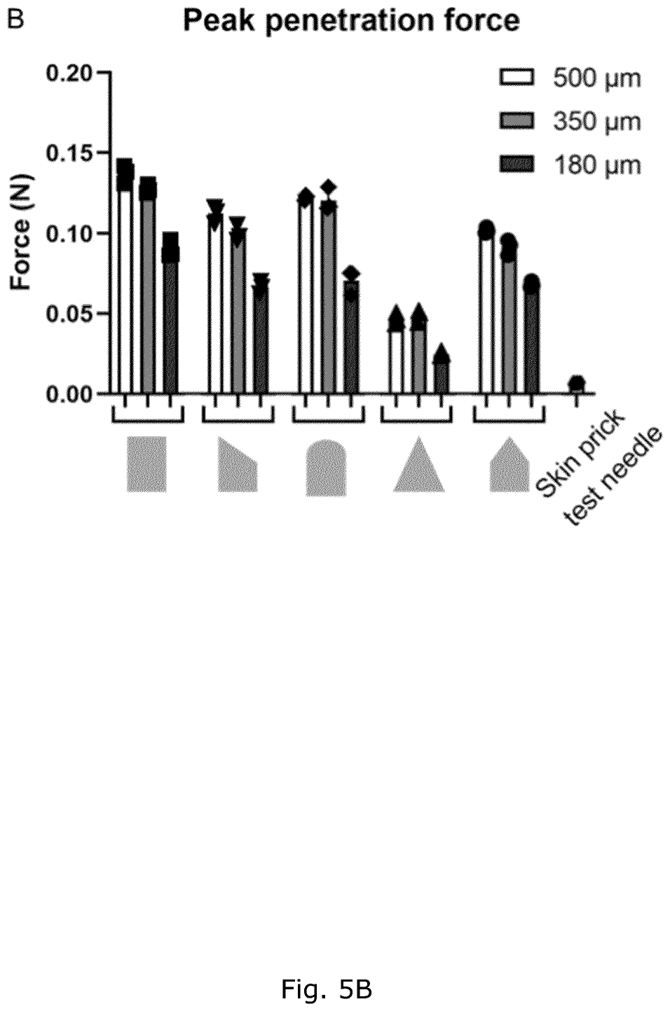

FIG. 5a shows a non-limiting example of the microneedles, wherein the microneedle is presented with different shapes.

In one embodiment, the shape of the microneedles is selected from the list consisting of flat, hypodermic, pencil, triangle and lancet.

In a preferred embodiment, the microneedles can be shaped as a triangle or a pencil, preferably a triangle, more preferably a pencil.

For the microneedles to be able to penetrate the skin and deliver the allergen to the mast cells the needles have to be of a length that enables correct delivery of the allergen.

Thus, in one embodiment, the of microneedles have a length of at least 400 μm, such as at least 500 μm, such as at least 600 μm, such as at least 700 μm, such as at least 800 μm, such as at least 900 μm, such as at least 1000 μm, such as at least 1100 μm, such as at least 1200 μm, such as at least 1300 μm, such as at least 1400 μm, such as at least 1400 μm, such as at least 1500 μm, such as at least 1600 μm, such as at least 1700 μm, such as at least 1800 μm, such as at least 1900 μm, such as at least 2000 μm, preferably microneedles have a length of at least 1000 μm or preferably microneedles have a length of at least 1300 μm.

In a preferred embodiment, the microneedles have a length in the range of 500-1000 μm, such as 600-1000 μm, such as 800-1000 μm, preferably the microneedles have a length of 1000 μm.

In another preferred embodiment, the microneedles have a length in the range of 500-2000 μm, such as 500-1800 μm, such as 500-1600 μm, such as 500-1400 μm, preferably the microneedles have a length of 1300 μm.

In another preferred embodiment, the microneedles have a length in the range of 500-2000 μm, such as 600-2000 μm, such as 800-2000 μm, such as 1000-2000 μm, preferably the microneedles have a length of 1500 μm. In particular for persons with relatively thick skin, and/or elderly persons, the needle may advantageously be relatively long.

In another embodiment, the microneedles have a length of 1000 μm.

### Loading of Antigen or Enzymes or Antibodies

The microneedles according to the invention can be loaded with different components depending on the use.

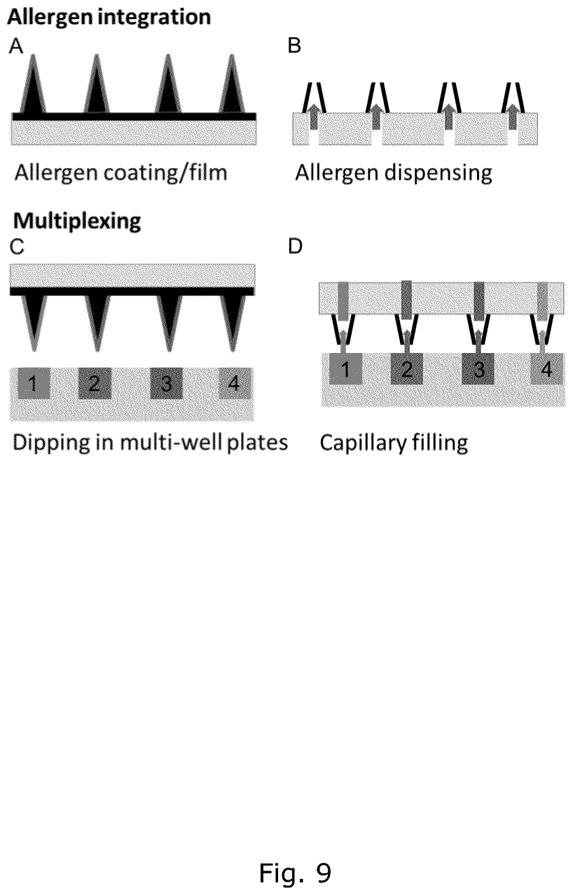

FIG. 9 shows four different embodiments on how the needles can be loaded with the different elements alone or in combinations, which is allergen and/or enzyme and/or biorecognition element.

In one embodiment, the elements can be pre-coated onto the needles as seen in FIG. 9a.

In another embodiment, the allergen can be dispensed through hollow needles as seen in FIG. 9b.

In a further embodiment, the microneedles can be coated by dipping the distal end of the needle into solution or pre-loaded wells as seen in FIG. 9c. In yet another embodiment, the needle can be filled by capillary filling as seen in FIG. 9d.

Another way of applying the allergen and/or enzyme and/or biorecognition element, alone or in combination, to the needles is by inkjet printing, which is a system where a small microdispenser is positioned above the microneedles and droplets of allergen are dispensed on the needles.

Thus, in one embodiment, the microneedles are coated by inkjet printing. In a preferred embodiment, the microneedles are coated by dipping the distal end of the needle into a solution.

In another embodiment, the at least one antigen is loaded onto the at least one microneedle on the microneedle array, prior to applying the microneedle array to the subject.

Each microneedle in the microneedle array can be individually loaded and thus, might contain either the same or different allergens, enzymes or biorecognition elements, alone or in combination.

Thus, in one embodiment all microneedles in the microneedle array are coated with different allergens and/or enzymes and/or biorecognition elements.

In another embodiment, all microneedles in the microneedle array are coated with the same allergen and/or enzyme and/or biorecognition elements.

In a further embodiment, the microneedles in the microneedle array are coated in groups of 2, such as 4, such as 5, such as 10, such as 20 with the same allergen and/or enzyme and/or biorecognition elements.

In another embodiment, the at least one allergen is positioned on the at least one microneedle.

### A Set of Parts

An aspect of the present invention relates to a set of parts for detecting an allergic reaction in the skin, the set of parts comprising:
allergen or allergens,
a microneedle array according to the invention, preferably anyone of claims 10-16,
at least one monitoring device for receiving, process and/or record the electrochemical signal detected by said microneedle array in the skin, said electrochemical signal being indicative for an allergic reaction in the skin.

### Use of the System

It may be mentioned that—in the context of the present invention—the said indications/informations are intended for assisting or guiding e.g. a clinician in making decisions of a therapeutic and/or a diagnostic character. Thus, the present invention is not designed to perform an actual diagnosis, merely providing information, i.e. indications/informations that may assist the clinician in performing the subsequent step of making the intellectual exercise of providing a diagnosis of the patient state and evaluation of the quality of the measurements, the diagnosis may then be followed by an action of therapeutic character, if needed.

Further, it may be mention that—in the context of the present invention—none of the embodiments or methods as described herein encompasses an invasive step representing a substantial physical intervention on the body which requires professional medical expertise to be carried out and which entails a substantial health risk.

The monitoring device as described herein, receives, processes and/or records the electrochemical signal as previously described and converts this electrochemical signal into data, which is stored on the monitoring device.

The data can be extracted and used elsewhere for different purposes.

In one embodiment, the data can be used to determine a risk of a subject having allergy.

An aspect of the present invention relates to a method for determining the risk of a subject having an allergy, wherein the method comprises
analysing a set of data previously obtained by going through the following steps, wherein the minimally invasive steps comprises
   contacting the skin of an individual with a microneedle array according to the present invention, wherein one or more allergens and/or enzymes and/or biorecgonition elements have been applied to the individual microneedles of the microneedle array,
   pressing the microneedles into the skin to expose the allergens to the interstitial fluid
   detecting one or more electrochemical signals with electrodes on the individual needles as a response to the introduced allergens and
   converting the obtained electrochemical signals into a set of data.

Another aspect of the present invention relates to a minimally invasive method for determining the risk of a subject having an allergy, wherein the method comprises.
analysing a set of data previously obtained by going through the following steps, wherein the minimal invasive steps comprises
   contacting the skin of a subject with the minimal invasive microneedle array according to the invention, wherein one or more allergens and/or enzymes and/or biorecgonition elements have been applied to the individual microneedles of the microneedle array
   expose the coated microneedles to the interstitial fluid and
   detecting one or more electrochemical signals with electrodes on the individual needles as a response to the introduced allergens

In on embodiment, the electrochemical signal is recorded and stored by a monitoring device according to the invention.

A further aspect of the present invention relates to a minimal invasive method for determining the risk of a subject having an allergy, wherein the method comprises
contacting the skin of a subject with the minimally invasive microneedle array according to the invention, wherein one or more allergens and/or enzymes and/or biorecognition elements have been applied to the individual microneedles of the microneedle array
expose the coated microneedles to the interstitial fluid and
detecting one or more electrochemical signals with electrodes on the individual needles as a response to the introduced allergens

In on embodiment, the electrochemical signal is recorded and stored by a monitoring device according to the invention.

### Determining the Risk of Having Allergy

Thus, one aspect of the invention relates to a minimally invasive method to determine an allergic reaction in the skin, the method comprising the step of
contacting the skin of an individual with a microneedle array according to the invention, wherein one or more allergens has been applied to the individual microneedles of the microneedle array,
pressing the microneedles into the skin to deliver the allergens to the interstitial fluid and
detecting one or more electrochemical signals with electrodes on the individual needles as a response to the introduced allergens.

In one embodiment, one or more allergens and/or one or more enzymes and/or one or more biorecognition elements have been applied to the needles before the needles enter the skin.

### Monitoring the Development of Allergy

As outlined above, the system according to the invention can be used to determine the risk of having allergy at a given time point. However, the method of the invention might also find use to monitor the development of allergy in a subject over time. Thus, another aspect of the invention relates to a method for monitoring the development of allergy in a subject, the method comprising
detecting a first electrochemical signal according to the method as described herein;
detecting a second electrochemical signal according to method as described herein, wherein the second electrochemical signal has been detected at a later time point than the first electrochemical signal;
comparing the first and second electrochemical signal;
wherein
   a lower electrochemical signal in the first detection compared to the second detection is indicative of a decreased level of allergy;
   an equal or higher level in the second detection compared to the first detection is indicative of unchanged or increased level of allergy.

In one embodiment, the system is used to monitor the development of allergy in a subject between a first electrochemical signal and a second electrochemical signal.

In one embodiment, a treatment against the allergy has taken place between the detection of the first electrochemical signal and the second electrochemical signal.

Thus, a further aspect of the invention relates to a method for monitoring the development of allergy in a subject, the method comprising analysing data extracted from a monitoring device according to the invention, wherein the data represents electrochemical signals obtained by the following steps:
detecting a first electrochemical signal according to the method as described herein;
detecting a second electrochemical signal according to method as described herein, wherein the second electrochemical signal has been detected at a later time point than the first electrochemical signal;
comparing the first and second electrochemical signal;
wherein
   a lower electrochemical signal in the first detection compared to the second detection is indicative of a decreased level of allergy;
   an equal or higher level in the second detection compared to the first detection is indicative of unchanged or increased level of allergy.

In one embodiment, the system is used to monitor the development of allergy in a subject between a first electrochemical signal and a second electrochemical signal.

In another embodiment, a treatment against the allergy has taken place between the detection of the first electrochemical signal and the second electrochemical signal.

In these embodiments, the invention may be partially implemented on corresponding data processing means including algorithms such as a computer program product comprising instructions which, when the program is executed by a computer, cause the computer to carry out these monitoring methods of development of allergy in a subject according to the invention.

This aspect of the invention is particularly, but not exclusively, advantageous in that the present invention may be accomplished by a computer program product enabling a computer system to carry out the operations of the apparatus/system of these monitoring aspects of the invention when down- or uploaded into the computer system. Such a computer program product may be provided on any kind of computer readable medium, or through a network.

### Effect of Treatment

It may also be advantageous to be able to monitor if a treatment for allergy is efficient. Thus, an aspect of the present invention relates to a method for determining the effect of a treatment protocol against allergy for a subject, the method comprising
detecting a first electrochemical signal according to the method as described herein;
detecting a second electrochemical signal according to method as described herein, wherein the second electrochemical signal has been detected at a later time point than the first electrochemical signal, wherein a treatment protocol has been initiated or completed before the detection of the first electrochemical signal or initiated, continued or completed between the detection of the first and second electrochemical signal,
comparing first electrochemical signal and the second electrochemical signal; wherein
   a lower electrochemical signal in the first detection compared to the second detection is indicative of a decreased level of allergy;
   an equal or higher level in the second detection compared to the first detection is indicative of unchanged or increased level of allergy.

Another aspect of the present invention relates to a method for determining the effect of a treatment protocol against allergy for a subject, the method comprising analysing data extracted from a monitoring device according to the invention, wherein the data represents electrochemical signals obtained by the following steps:
detecting a first electrochemical signal according to method as described herein;
detecting a second electrochemical signal according to method as described herein, wherein the second electrochemical signal has been detected at a later time point than the first electrochemical signal, wherein a treatment protocol has been initiated or completed before the detection of the first electrochemical signal or initiated, continued or completed between the detection of the first and second electrochemical signal,
comparing first electrochemical signal and the second electrochemical signal; wherein
   a lower electrochemical signal in the first detection compared to the second detection is indicative of a decreased level of allergy;
   an equal or higher level in the second detection compared to the first detection is indicative of unchanged or increased level of allergy.

In one embodiment, the treatment is a treatment effective against allergy.

In another embodiment, the treatment is allergen immunotherapy.

In another embodiment, the electrochemical signalling is detected by a monitoring device.

In a further embodiment, the electrochemical signal can be detected after the microneedle array has been applied to the skin for 40 min, such as 20 min, such as 10 min, such as 5 min.

In another embodiment, the subject is a mammal, preferably a human.

The embodiments of the invention related to monitoring if a treatment for allergy is efficient may of course also be implemented partially on corresponding data processing means including algorithms such as a computer program product comprising instructions which, when the program is executed by a computer, cause the computer to carry out these monitoring methods of a treatment of allergy in a subject is effective according to the invention.

### EXAMPLES

### Example 1—Setup for Allergy Testing

Microneedle-based electrochemical sensors are fabricated and used to introduce allergens into the skin tissue. If the patient is sensitive to the specific allergen, mast cells in the interstitial fluid are activated by the allergens and release biomarkers such as histamine. Three main sensor configurations are considered for the quantitative electrochemical measurement of the released biomarkers (FIG. 2):
Configuration 1: Direct electrochemical sensing in the skin (FIG. 2A): The biomarkers released by the mast cells are directly oxidized or reduced at the electrode surface using amperometric or voltammetric methods.

Detection of histamine using fast scan cyclic voltammetry (FSCV) has been shown (Puthongkham 2019) where histamine oxidation was observed at a potential of approx. 1.1 V.
Configuration 2: Electrochemical enzymatic biosensing in the skin (FIG. 2B): Enzymes immobilized on the electrode surface convert the biomarkers released by the mast cells into an intermediate product. The product is then either measured directly or after further conversion by additional enzymes or non-enzymatic catalysts.

For example, histamine is converted by diamine oxidase (DAO) into H₂O₂, which then is detected electrochemically using peroxidase enzymes or Prussian Blue as electrocatalyst, as shown in example 2.
Configuration 3: Electrochemical ELISA-based immunosensing outside the skin (FIG. 2C): Biorecognition elements immobilized on the electrode surface capture the biomarkers released by the mast cells. The microneedle electrodes are retrieved from the skin and solution is added containing a detection antibody completing the ELISA-based immunosensor with a sandwich bioassay on the electrode surface. The detection antibody is labelled with an electroactive label (e.g. methylene blue, anthraquinone), or an enzyme label catalyzing the production of an electroactive product (e.g. alkaline phosphatase, horseradish peroxidase) (Kondzior 2020). The amount of electroactive labels is then quantified by electrochemical sensing, allowing to conclude on the concentration of analyte present in the skin.

For example, tryptase is captured in the skin by tryptase antibodies immobilized on the electrode surface. Outside the skin, a tryptase detection antibody labelled with an electroactive label is added and the amount of tryptase bound to the electrode surface is quantified based on the electrochemical signal recorded from the electroactive label.

### Example 2—Measurement of an Allergen in Mast Cell Medium

### Aim

The overall aim was the development of an electrochemical sensing method with the prospect of detecting histamine released by mast cells in the interstitial fluid of the human skin tissue. Pyrolytic carbon electrodes were selected as the transducer because this is one of the proposed materials for microneedle-based electrochemical sensing. 3D pyrolytic carbon pillar electrodes were applied for the sensor development, because their properties (surface area, conductivity) and size are similar to an array of out-of-plane carbon microneedles in the final application.

More specifically, the goal of this study was to develop a Prussian blue (PB)-based enzymatic histamine biosensor with capabilities to detect histamine in mast cell medium. In previous work (Tehrani-2021), we demonstrated the amperometric detection of H₂O₂ in buffer in the μM- and sub-μM range with a 3D PB-modified pyrolytic carbon sensor. PB electrodeposition on the 3D pyrolytic carbon electrodes was optimized and reproducible sensors were obtained. In the current study, we applied the same type of transducer (PB-modified 3D pyrolytic carbon electrode) to introduce a novel electrochemical method for measuring histamine. For this purpose, an enzyme layer was immobilized on the PB film serving as biorecognition element converting histamine in the sample into H₂O₂, which then was measured due to PB-mediated reduction at the electrode surface. The method is based on a new concept relying on charge-accumulative potentiometry combined with chronoamperometry as the electrochemical measurement technique.

### Material and Method

### Reagents

The chemical reagents required for PB modification of the electrodes including potassium hexacyanoferrate (III), iron (III) chloride, potassium chloride, hydrochloric acid 37%, monobasic potassium phosphate and dibasic potassium phosphate were all purchased from Sigma, and the stock solutions were prepared freshly using double distilled water before each experiment. For the initial proof-of-concept experiments, H₂O₂ 35% was obtained from Sigma and diluted in phosphate buffer. For immobilization of an enzyme layer, bovine serum albumin (BSA) lyophilized powder ≥96% and glutaraldehyde (GA) solution grade I, 70% in H₂O from Sigma were dissolved/diluted in phosphate buffered saline (PBS) pH 7.2 and double distilled water, respectively. Plant-derived diamine oxidase (DAO) solution with enzymatic activity of 2672 U mL⁻¹ was purchased from IBEX Pharmaceuticals Inc., Canada and used as it was supplied. For in vitro histamine detection in the mast cells medium, the supernatant of the activated LAD2 human mast cells containing degranulated histamine at different concentrations were provided by the allergy clinic at Copenhagen university hospital in Gentofte, Denmark. LAD2 mast cell titration and activation were performed in histamine release buffer (HR-buffer) composed of PIPES (RefLab) and 0.5% human serum albumin (CSL Behring, USA). Compound 48/80 (1 mg mL⁻¹, dissolved in sterile water) from Sigma Aldrich was used to induce mast cell degranulation.

### Electrochemical Measurements

All electrochemical measurements were performed using a MultiPalmSens4 potentiostat and MultiTrace 4.3 software was used to acquire data. Excel and OriginPro 2018 were employed for data treatment and statistical analysis of the obtained results. An external silver wire covered with silver electrode (Ag/AgCl) in 3M KCl solution purchased from ItalSens was used as reference electrode (RE). A double-sided Pt coated chip was used as counter electrode (CE).

### Electrode Modification and Prussian Blue Modification

A 3D pyrolytic carbon electrode consisting of 284 micropillars with a height of 225 μm and a diameter of 68 μm (total geometrical surface area of 26.2 mm²) and metal (Ti+Au) leads patterned on a silicon chip was used as working electrode (WE). A detailed description of the electrode fabrication process can be found in our previous work (Tehrani-2021). Four oxygen-plasma treated WEs were modified simultaneously with PB by applying a potential of 444 mV for 80 s to the WEs immersed in a fresh PB growth solution containing 2.5 mM K₃Fe(CN)₆, 2.5 mM FeCl₃, 0.1 M HCl and 0.1 M KCl. Thereafter, the electrodeposited PB films were activated in a solution consisting of 0.1 M HCl and 0.1 M KCl by sweeping the potential between 0.4 and −0.1 V (starting at 0.4 V) at a scan rate of 50 mV s⁻¹ for 25 cycles. Next, the PB-modified electrodes were rinsed with 0.1 M HCl and were annealed at 100° C. for 1 hour. Finally, the PB films were conditioned and stabilized at −6 mV for 600 s followed by 25 cycles between +0.4/−0.1 V at 50 mV s⁻¹ in phosphate buffer containing 0.05 M K₂HPO₄/KH₂PO₄ and 0.1 M KCl with pH adjusted to 5.5 using HCl.

### Enzyme Immobilization

DAO was immobilized on the PB-modified 3D pyrolytic carbon WE together with BSA as a spacer molecule and GA as a cross-linker. A concentration of 10 mg mL⁻¹ was obtained for the DAO stock solution by protein spectrophotometry. For the immobilization, the enzymes were used undiluted in the buffer in which the stock was supplied. To avoid repetitive thaw and freeze cycles, DAO stock solution was aliquoted immediately when received from the supplier and the aliquots were stored at −70° C. 2 μL of DAO solution (10 mg mL⁻¹) were gently mixed in a PCR tube with 1 μL of 0.5% BSA freshly prepared in PBS pH 7.2. Right before immobilization, 1 μL of 1% GA (diluted in distilled H₂O) was added to the DAO/BSA in the tube and mixed by gentle pipetting without creating air bubbles. Eventually, 4 μL of the DAO/BSA/GA mixture were drop cast on the WE and left at room temperature (22-25° C.) for 30 minutes to immobilize. The immobilization and cross-linking process was finished by gently rinsing the electrode with PBS pH 7.2 three times to remove the unbound molecules.

### Measurement Concept and Sequence

First a step of a reducing potential was applied by chronoamperometry to the PB/DAO-modified WE electrode to convert PB to PW analogous to charging a capacitor. PB or ferric ferrocyanide (Fe₄³⁺[Fe²⁺ (CN)₆]₃) crystal unit cell contains low spin iron (Fe²⁺) and high spin iron (Fe³⁺) with carbon and nitrogen (cyanide) bonds in between. The open framework of PB has some interstitial sites and vacancies where counter cations such as potassium (K⁺) and small molecule of H₂O₂ can be intercalated. Fe³⁺ ions make up of almost half of the total amount of Fe atoms in the PB unit cell ([Fe³⁺][Fe^{2+/3+}]⁻¹=0.5)[11]. The PB conversion to PW involves reduction of Fe³⁺ ions to Fe²⁺ by drawing electrons from the electrode. In the second step, the electrical circuit was disconnected to avoid the flow of charge through the electrode and the sample containing histamine was added to the measurement solution while the potential of the WE vs. RE was measured by open circuit potentiometry. As depicted in equation (1), DAO catalyzes the oxidation of histamine to imidazole-4-acetaldehyde, ammonia (NH₃) and H₂O₂ in the presence of molecular oxygen dissolved in the aqueous solution (reaction not shown in the schematics). At open circuit, H₂O₂ oxidizes PW to PB (equation 2) meaning that Fe²⁺ ions are oxidized to Fe³⁺ by losing electrons analogous to discharging the capacitor. This imposes a positive shift in the potential of the WE. The oxidation of a unit cell of PW to PB is a four-electron process [12], while the catalytic reduction of each H₂O₂ molecule to hydroxide ions (OH⁻) involves two electrons [9]. Therefore, for the oxidation of each PW unit cell, two H₂O₂ molecules are consumed and four hydroxide ions produced. Unlike the conventional amperometric detection of PB-catalyzed H₂O₂ reduction, in which charge passes through the electrode as it is produced by the reaction, in this technique the charge (positive-charge) is accumulated at the electrode for the open circuit.

With the reconnection of the circuit, the accumulated charge was measured by another step of chronoamperomtery. The main hypothesis of this electrochemical transduction concept is that the sudden measurement of the accumulated charge provides more sensitivity to traces of analyte in nM range and enhances the signal. A reducing potential was applied to regenerate the PW film (equation 3) and the corresponding reduction current was recorded. Regeneration of PW is analogous to recharging the capacitor, as electrons are drawn from the electrode to reduce the Fe³⁺ ions previously produced as a result of PW film reaction with H₂O₂. The integral of the measured current yielded the accumulated charge which corresponded to the amount of H₂O₂ produced by the enzymatic reaction. Eventually, because the concentration of H₂O₂ is directly proportional to the concentration of histamine, the biosensor can be used to quantify the amount of histamine in a sample of unknown concentration.

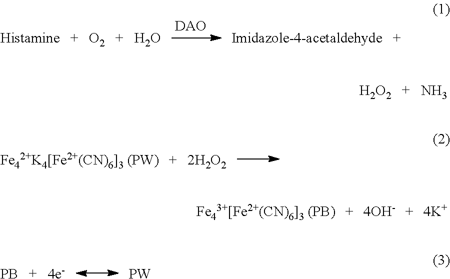

### Histamine Detection in Buffer

Histamine samples were prepared and tested in the following buffer conditions:
Phosphate buffer (0.05M K2HPO4/KH2PO4) containing 0.1 M KCl pH 5.5/RT (optimal for Prussian blue)
Phosphate buffer containing 0.1 M KCl pH 7.2/37° C. (optimal condition for DAO enzyme)

The histamine concentrations were 100 nM, 500 nM, 1 μM, 10 μM, 100 μM, 1 mM to identify the dynamic range and the optimal measurement conditions. Histamine measurements were conducted in separate glass beakers using the identical three steps as described above:
(1) Chronoamperometry with a sampling interval of 1 s, duration 60 s, E: 0.0 Volt, to regenerate PW; (2) OCP with a sampling interval of 1 s, duration 120 s where the histamine sample was added at the start of OCP, (3) chronoamperometry with a sampling interval of 1 ms, duration 10 s, E: 0.0 Volt, to measure Histamine reaction; before the measurements with histamine, 3-5 cycles of baseline measurements were performed until a stable baseline was obtained

### Histamine Detection in Cell Supernatant

Method: Chronoamperometry (sampling interval: 1 s, duration 60 s, E: 0.0 Volt, to regenerate PW)+OCP (sampling interval: 1 s, duration 300 s, Histamine sample will be added at the start of OCP)+Chronoamperometry (sampling interval: 1 ms, duration 10 s, E: 0.0 Volt, to measure Histamine reaction) including 3-5 cycles of baseline or until a stable baseline is obtained.

### Results

Preliminary measurements with histamine in buffer solutions showed that histamine measurements in sub μM concentrations were possible with the proposed sensing method.

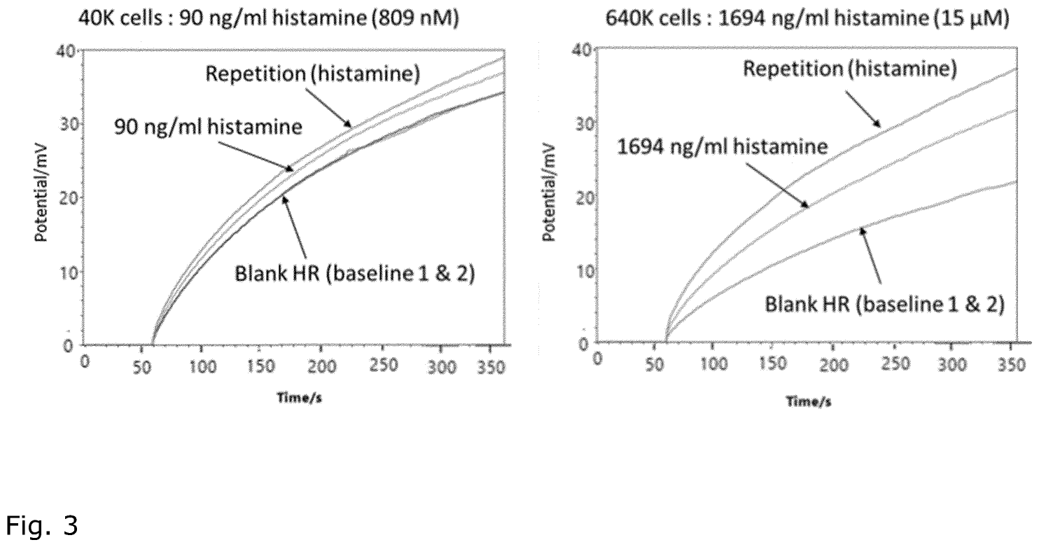

Therefore, further measurement were performed with mast cell supernatant containing different concentrations of histamine. FIG. 3 shows the results obtained for measurements with two different concentrations. For repetitive measurements without histamine present, the signal remained identical. However, upon introduction of the supernatant, an increase in the potential was observed due to the conversion of the histamine to H₂O₂. For higher histamine concentration, the increase in potential was larger.

### Conclusion

In this first proof-of-concept of the novel sensor method, a clearly distinguishable response from 800 nM histamine in cell supernatant was recorded.

### Example 3—Manufacturing of In-Plane Microneedle-Based Electrochemical Sensors and Out of Plane Pyrolytic Carbon Microneedles

### Aim

The aim was to fabricate Si-based in-plane microneedles with a length of 500-1000 μm, a thickness of 180 μm and a width of 200-400 μm. Pyrolytic carbon microelectrodes were integrated on the top side of the microneedles to allow for electrochemical sensing. Each sensor chip included an array of 3 in-plane microneedles.

Another aim of this study was to fabricate out of plane pyrolytic carbon microneedles with a length of approximately 1000 μm and with scaffold like geometry to provide high surface area for electrochemical sensing. Polymer microneedles were designed and fabricated using 3D printing and subsequently converted into carbon through a pyrolysis process.

### Material and Methods

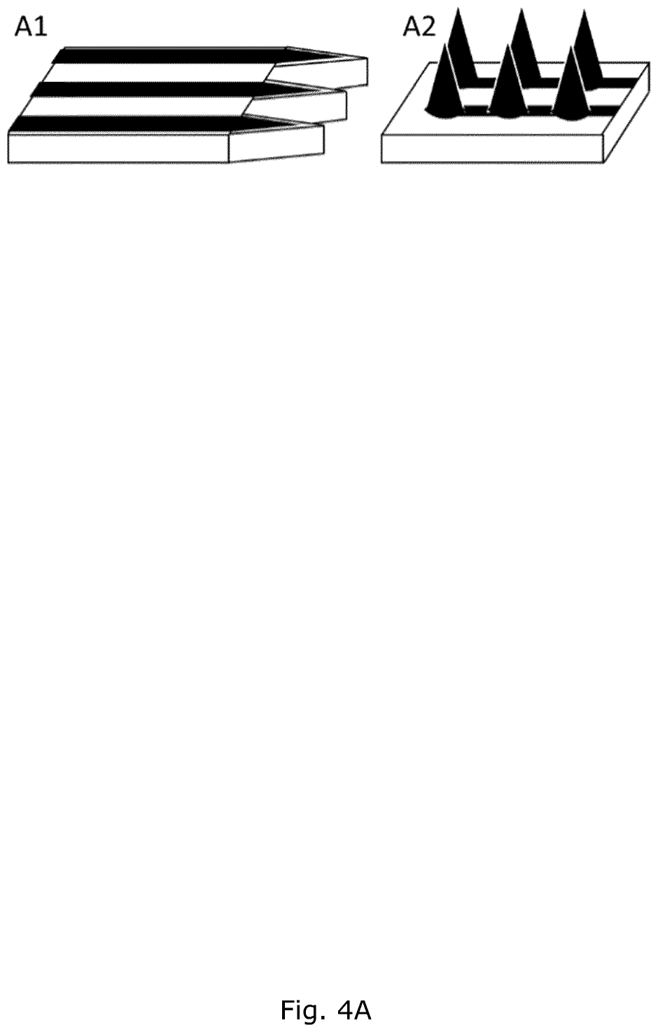

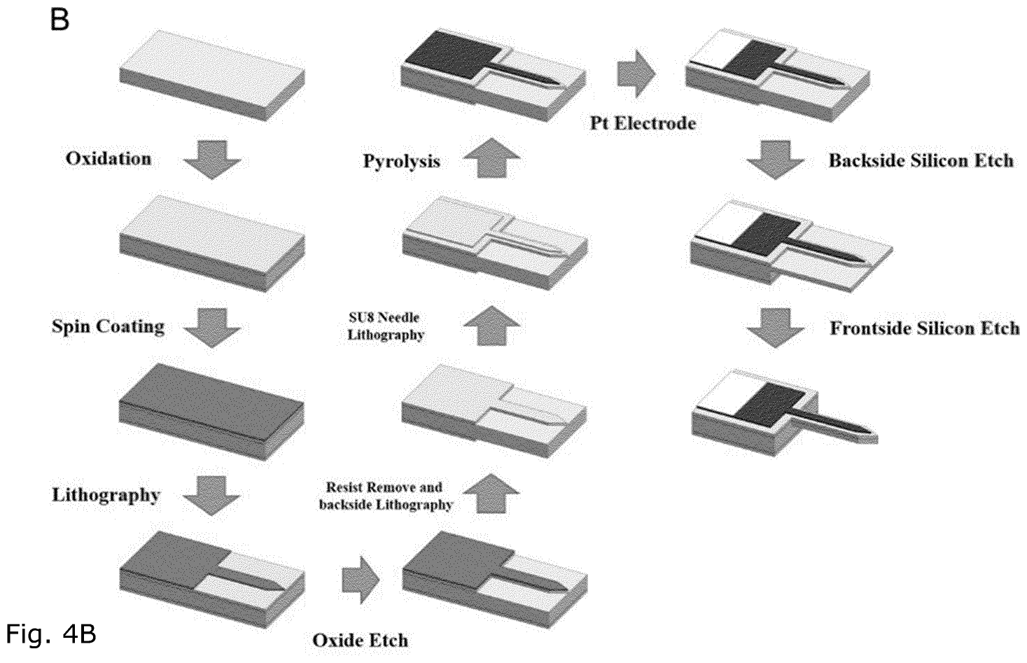

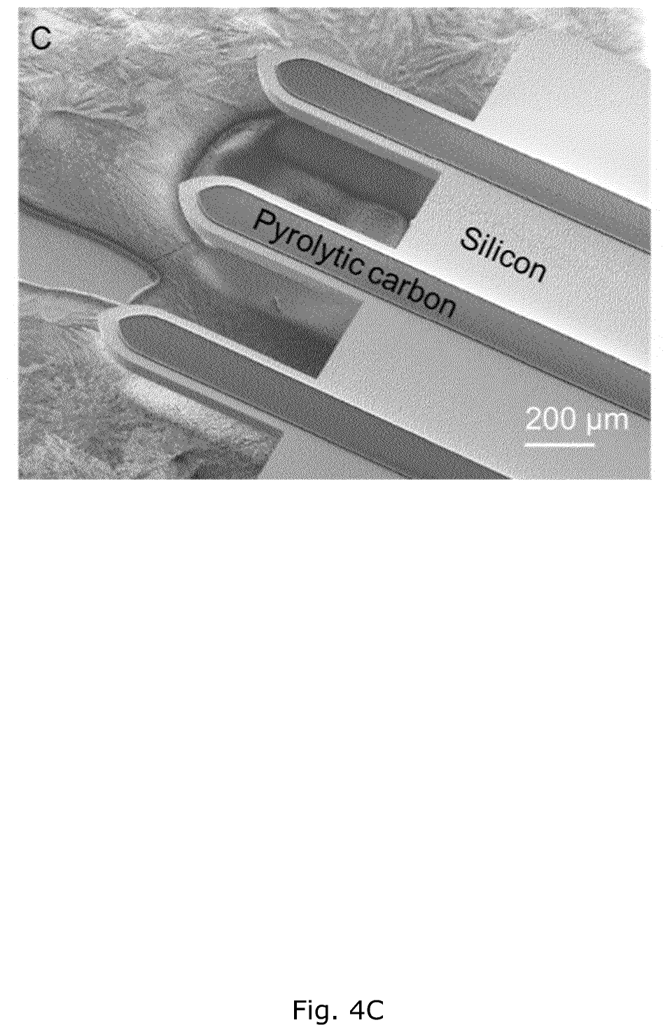

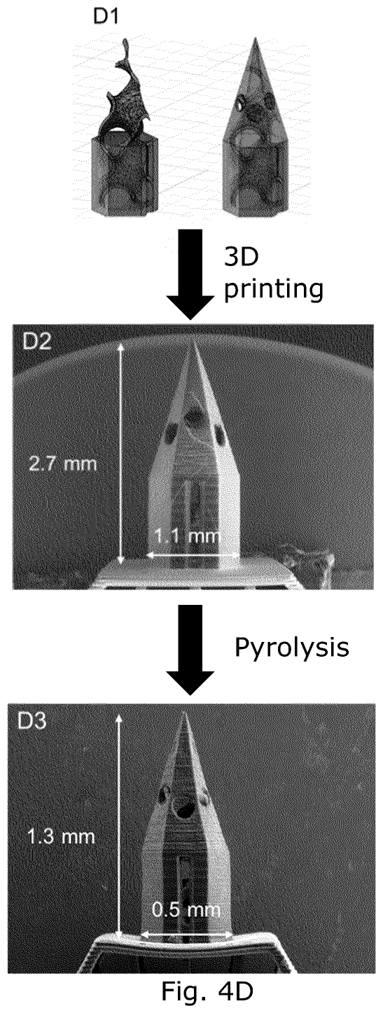

FIG. 4B schematically illustrates the fabrication process of the Si-based in-plane microneedles. First, 600 nm SiO₂ were deposited on 4-inch Si wafers by wet oxidation at 1050° C. Next, a photolithography step on the front side of the wafer was performed using 1.5 μm thick photoresist patterned on a maskless aligner to define the outline of the in-plane microneedles and the Si chip. The photoresist was used as an etch mask to transfer the pattern into the SiO₂ by reactive ion etching (RIE). A second step of UV photolithography was conducted on the backside of the wafer followed by another RIE process to etch the SiO₂. For the definition of the pyrolytic carbon microelectrodes, the negative epoxy photoresist SU-8 was spin coated on the front side of the Si wafer and patterned by UV photolithography. The SU-8 photoresist was then converted into pyrolytic carbon by heating to 900° C. in inert nitrogen atmosphere in a PEO-604 multipurpose furnace (ATV Technologies GmbH, Germany). For enhancement of the electrical contact between carbon electrodes and the external measurement setup, Pt contact pads were deposited on the Si chip by e-beam deposition through a shadow mask. This was followed by a deep reactive ion etching (DRIE) process from the backside defining the outline of the Si chip and the final thickness of the in-plane Si microneedles. Finally, the wafer was placed on a carrier wafer by crystal bonding and the outline of the Si chip and the Si in-plane microneedles was etched from the frontside by DRIE.

For fabricating the out of plane pyrolytic carbon microneedles, 3D polymer microneedles with a height of approximately 2 mm were designed using Fusion360 3D design software as shown in FIG. 4D1. The internal structure was semi-hollow, allowing access to electrolyte while still providing mechanical stability. The polymer microneedles were fabricated by microprojectionstereolithography (μPSLA) with a commercial 3D printing resin in a BMF S140 3D printer. After printing, the structures were washed in isopropanol and cured with UV light to enhance the mechanical stability. The polymer microneedles were pyrolyzed in a quartz tube furnace at 900° C. in nitrogen atmosphere.

### Results

FIG. 4C shows a Si chip with an array of 3 in-plane Si microneedles. Pyrolytic carbon electrodes are integrated on each individual microneedle, allowing for electrochemical measurements.

FIG. 4D2 shows the polymer microneedles after 3D printing. The height is approximately 2 mm and the internal and external structures are well defined.

FIG. 4D3 shows the corresponding carbon microneedles after pyrolysis. The structures shrink with about 50% in all direction resulting in a final needle height of approximately 1 mm. However, internal and external structures are well preserved. Also, the mechanical stability of the carbon microneedles was successfully tested. Moreover preliminary testing showed that the carbon microneedles could be used as electrodes for electrochemistry.

### CONCLUSION

This example demonstrates that Si-based in-plane microneedles with a length of 500-1000 μm, a thickness of 180 μm and a width of 200-400 μm can be fabricated along with out of plane pyrolytic carbon microneedles with a length of approximately 1000 μm. The out of plane pyrolytic carbon microneedles have a scaffold like geometry that provide high surface area for electrochemical sensing.

### Example 4—Mechanical Stability and Skin Penetration

### Aim

The aim was to test Si-based in-plane microneedles (MNs) with different dimensions and shapes in terms of their mechanical stability and ability to penetrate the epidermis of the skin using a skin simulating in vitro hydrogel model. Finally, rat skin samples were used for the same measurements and histology was performed to investigate penetration depth and tissue damage.

### Material and Methods

Si chips with arrays of 3 in-plane Si microneedles (IPSMN) were fabricated with a process similar to the one described in example 3. The mechanical stability and penetration force of the IPSMN were tested using 1.5% w/v agarose gel in a 3D printed mold serving as a skin simulating hydrogel. The penetration forces were measured for IPSMN of different designs and dimensions with a custom-made 3D printed holder mounted on a texture analyzer (TA.XTplusC, Stable Micro Systems). Five MN tip designs were fabricated for this purpose: Flat (control), hypodermic, pencil, triangle, and lancet (FIG. 5A). The IPSMN underwent several iterations for optimization of their dimensions to ensure easy skin penetration while maintaining their mechanical stability. The different MN tip shapes were investigated together with MN lengths (L) of 500 μm, 750 μm and 1000 μm. The thickness (t) of the IPSMN was 500 μm, 350 μm and 180 μm. The width (w) was either 200 μm or 400 μm.

An animal study was conducted to examine the mechanical stability and penetration force of IPSMN in skin samples. It was decided to investigate the tip shapes flat (for control), triangle and pencil with L=500 μm or L=1000 μm, thickness t=180 μm and width w=400 μm or w=200 μm. For the animal experiment, 12 naïve Brown Norway (BN) rats were used and sacrificed before performing penetration with the IPSMN. Abdominal skin samples of approximately 30 mm×30 mm from six BN rats were used for penetration force measurements.

The skin samples were removed and fixated in their original size on a subjacent supporting layer of hydrogel. The remaining six BN rats were after euthanization skin pricked with the IPSMN on their abdomen and skin samples were excised from these areas for histology analysis to determine penetration depth and potential skin damage. Animal experiments were carried out at the Technical University of Denmark's facilities. Ethical approval was given by the Danish Animal Experiments Inspectorate with the authorization number (2020-15-0201-00732-C1). The experiments were overseen by the University's in-house Animal Welfare Committee for animal care and use.

### Results

The texture analyzer was able to measure a difference in penetration force between the different tip designs and MN dimensions upon penetration of the hydrogel-based in vitro skin model. None of the IPSMN were damaged during the experiments, demonstrating their mechanical stability. As expected, more narrow microneedles (w=200 μm) and thinner microneedles required less force for skin penetration. FIG. 5B shows the peak penetration force extracted for the array of 3 IPSMN with variable MN tip design in 1.5% w/v agarose gel as the skin simulating hydrogel for IPSMN with a length of 500 μm, a width of 400 μm and variable thickness. Furthermore, it was observed that the triangle tip required the lowest penetration force, comparable to the commercially available skin prick test, considering that the force should be divided by a factor 3 due to the presence of 3 microneedles in the array.

For penetration force measurements in the animal study, it was observed that flat MN tips and MNs with L=500 μm were not able to penetrate the rat skin. For IPSMN with L=1000 μm and other shapes than flat, results were in agreement with the in vitro tests, identifying the triangular microneedles as the ones requiring the lowest penetration force.

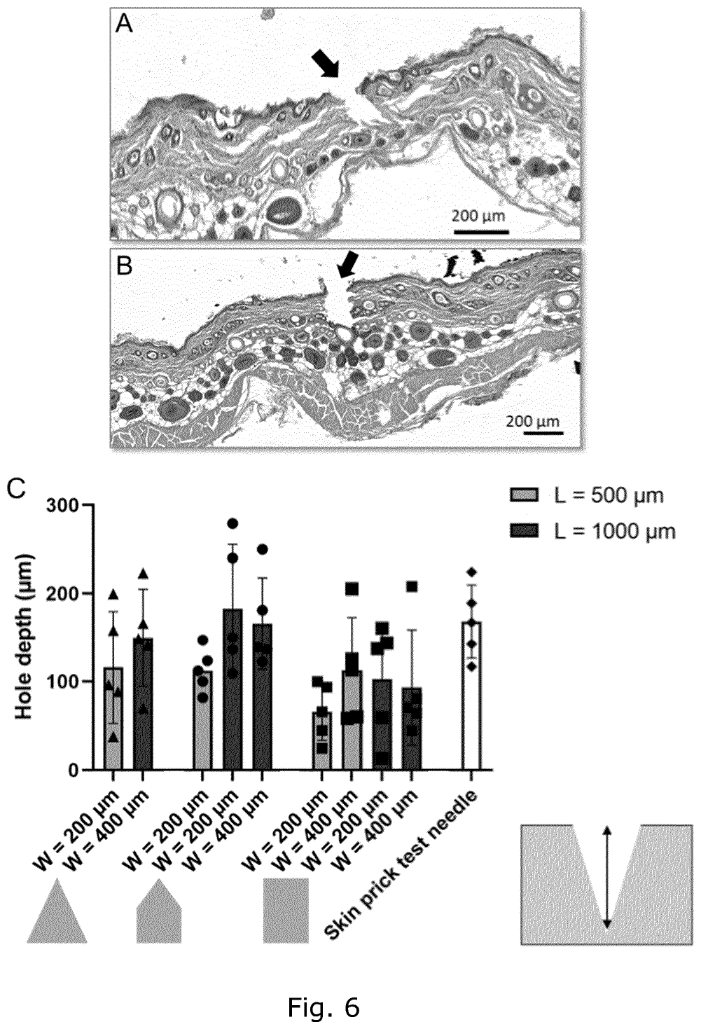

FIG. 6A-B show histology cross-sectional slides of rat skin stained with Mayer's hematoxylin and eosin where a MN penetration hole is identified. Quantitative analysis of the tissue damage (FIG. 6C) indicated that the IPSMN with L=500 μm and the ones with L=1000 μm and a flat tip were not able to penetrate as deep as the commercially available skin prick test. In comparison, the IPSMN with L=1000 μm and triangular/pencil tip showed comparable hole depth as the skin prick test. At the same time, these IPSMN induced less lateral damage to the skin tissue.

### Conclusion

It was concluded that IPSMN with a length L=1000 μm are required for sufficient penetration of rat skin. In general, the experiments indicate that IPSMN with w=200 μm and a triangular tip shape require the lowest penetration force while inducing the least tissue damage. However, for electrochemical measurements large surface area is an advantage, which means that pencil shaped IPSMN with w=400 μm could be a good compromise.

### Example 5-Microneedle Insertion in Pig and Human Skin for Optimization of the Insertion Time

### Aim

The aim of this study was to demonstrate the dermal delivery of a fluorescent model compound in pig and human skin samples using microneedles and to determine the optimal insertion time.

### Materials and Methods

Frozen human and pig skin samples were obtained and defrosted for a few hours. The fat was removed from the skin tissue on the dermis side and the samples were fixed on a cork plate wrapped in tin foil using pin needles. In-plane Si microneedles with a length of 1000 μm, thickness of 180 μm and width of 400 μm were coated with Rhodamin B by dip coating. The microneedles were inserted into the skin tissue with a force of 5 N for a duration of 2, 30 or 60 s. The insertion area was removed with a biopsy punch and prepared for cryostat histology. The samples were sliced with a thickness of 30 μm and analyzed using a fluorescence microscope.

### Results

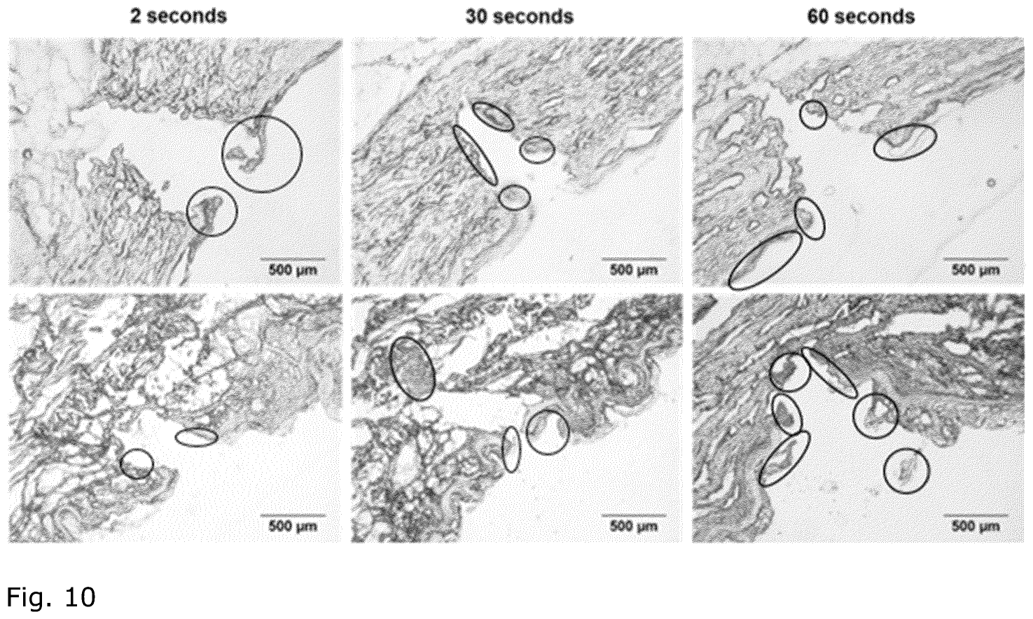

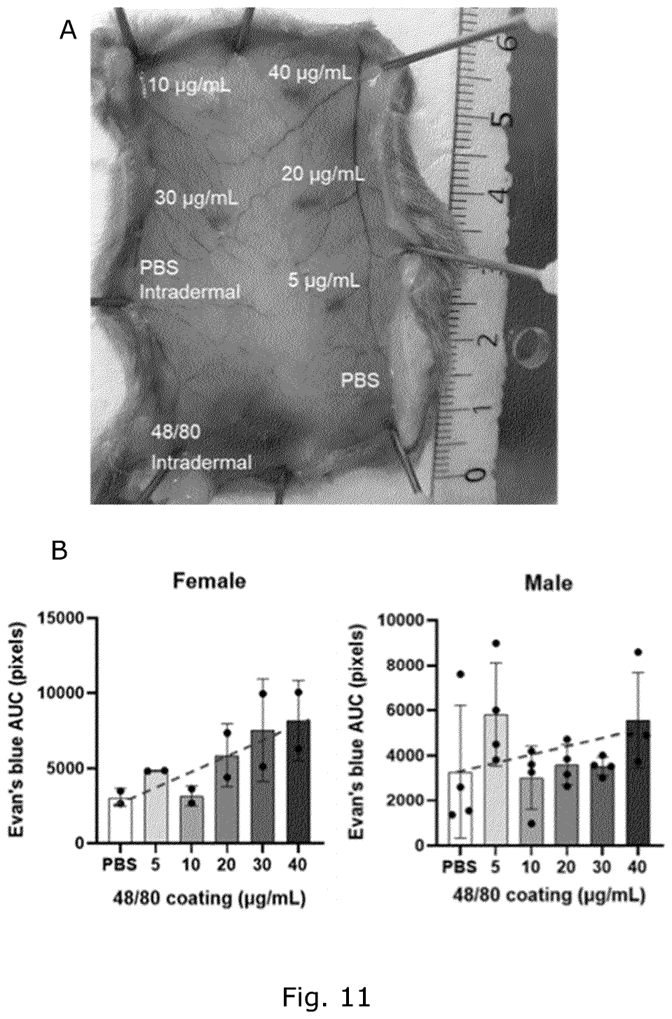

Histology images of the biopsies of pig and human skin samples with punctures from microneedle insertion for 2 seconds, 30 seconds or 60 seconds are shown in FIG. 10. The images show the presence of Rhodamin B (black circles) at the insertion point and demonstrate the successful delivery of the model compound namely Rhodamin. An insertion time of 30-60 seconds was identified as suitable for delivery.

### Conclusion

This study demonstrates that the microneedles can deliver a model compound to human and pig skin samples when inserting said microneedles into the skin surfaces. In addition, an insertion time of 30-60 seconds was identifired as suitable for delivery of the model compound.

### Example 6—Dermal Delivery of Compound 48/80 in Brown Norway Rats Aim

The aim of this in vivo study was to determine the capability of coated microneedles to penetrate skin tissue, deliver a compound and activate the mast cells in the interstitial fluid, thereby validating the microneedles as a dermal delivery system. The compound 48/80 is used for this purpose as a positive control because it activates all the mast cells regardless of whether they are sensitized or not.

### Materials and Methods

In-plane Si microneedles with a length of 1000 μm, a thickness of 180 μm and a width of 400 μm were coated with different concentrations of 48/80 dissolved in 1 wt. % solution of carboxymethylcellulose (CMC) in PBS solution (pH 7.4) using dip coating with a texture analyzer. Brown Norway rats were sedated and shaved on the abdominal skin. On each animal eight different penetration areas were marked. After this, 2 μL/g body weight of 1% Evan's blue dissolved in PBS was injected in the tail vein of the rats. Evan's blue binds to albumin in the blood and colors it blue. An immological reaction in the skin such as the one expected due to mast cell degranulation induces increased blood circulation and accumulation at the site where it takes place, resulting in local blue coloration of the tissue. The coated microneedles were manually inserted in the rat skin for 60 s. Two intradermal injections were included in each animal as positive (48/80) and negative (PBS) controls. The animals were sacrified after 15 min. The skin was excised, stretched to its original size and fixed on Styrofoam using pin needles. Imaging of the skin was performed with a mobile phone camera followed by image analysis to determine the blue coloration (shown as a black coloration in FIG. 11A) in the area around the needle injection point.

### Results

The results for dermal delivery of the compound 48/80 and PBS as a negative control in brown Norway rats are shown in FIG. 11. FIG. 11A shows a picture of the rat skin, wherein the different concentrations of compound 48/80 and PBS are indicated and a black marking is visible for some of the concentrations of 48/80. The coloration of the rat skin for every concentration of compound 48/80 and PBS has been quantitized for female and male rats in FIG. 11B. The results show a tendency for increased immunoresponse for increasing concentrations of 48/80, indicating a successful delivery of the compound to the rat skin.

### Conclusion

The results show a tendency for an increased immunoresponse for increasing concentrations of compound 48/80 which indicates that a successful delivery of the compound to the rat skin can be achieved using the microneedles.

### Example 7—Measuring an Allergic Response in Rats Aim

The aim of this study was to investigate the capability of coated microneedles to penetrate skin tissue, deliver a compound (birch pollen extract (BPE), bet v1 birch allergen, 48/80, PBS)) and activate mast cells indicative of the induction of an allergic response to birch pollen extract or bet v1 birch allergen in naive and sensitized rats. Bet v1 is a protein also included in BPE and the most common compound that causes birch pollen allergies. The coated microneedles were compared to intradermal injection of said compounds.

### Materials and Methods

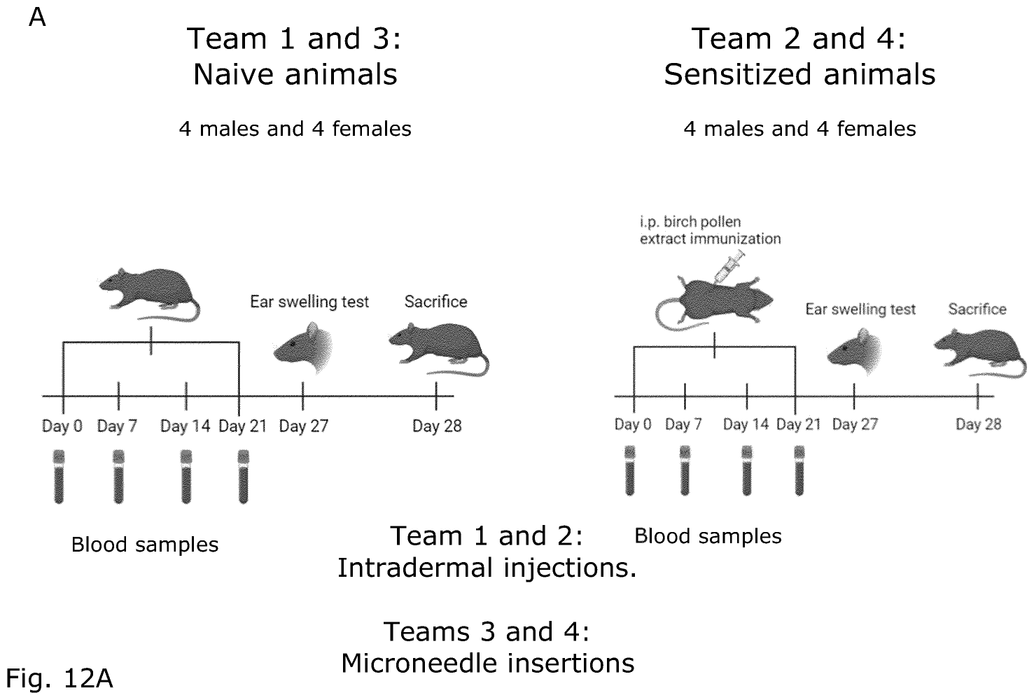

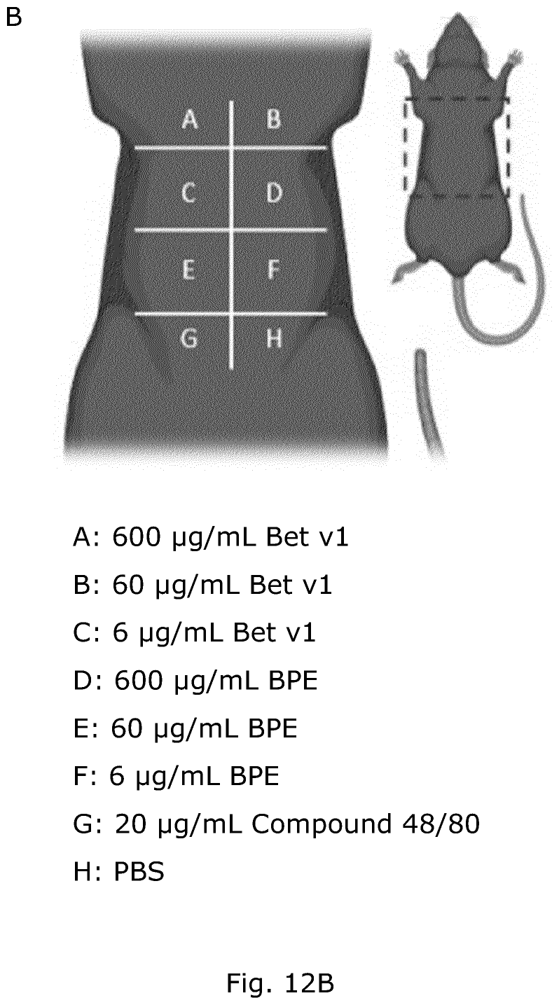

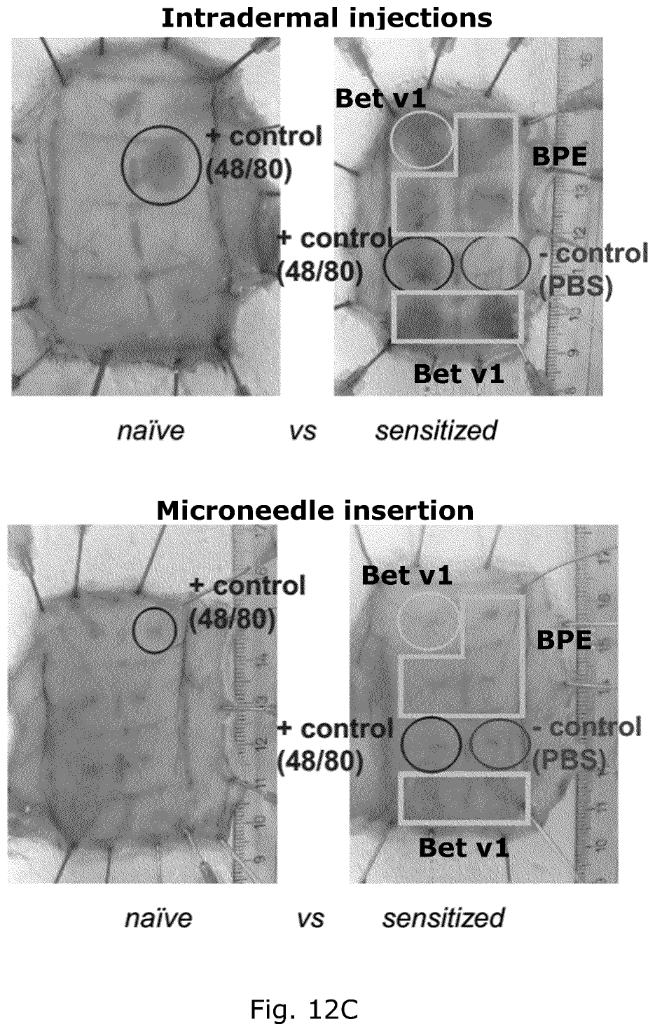

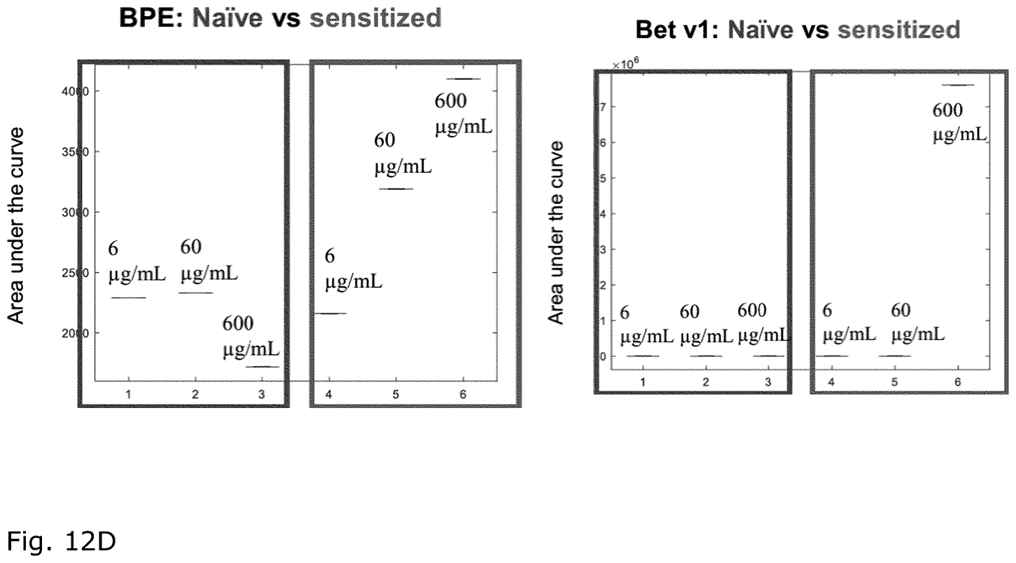

Brown Norway rats were allocated into four groups with teams 2 and 4 being sensitized rats (n=8/group, 4/gender, 4-6 weeks of age) and teams 1 and 3 being naive rats (n=8/group, 4/gender, 4-6 weeks of age). Therefore, thirty-two rats were used in total for this animal experiment. The experimental setup is illustrated in FIG. 12A. Teams 1 and 2 received intradermal injections whereas teams 3 and 4 received microneedle insertions. The sensitization in teams 2 and 4 was performed by injecting 50 μg BPE in 0.5 ml PBS by intraperitoneal (IP) injection whereas the naive rats received PBS (negative control). Immunizations occurred once a week for four consecutive weeks (Day 0, 7, 14 and 21). Blood samples were collected from all animals both naive and sensitized animals before immunization (Day 0, 7, 14 and 21). After the last IP post-immunization, the rats were subjected to an ear swelling test (EST) with 10 μg BPE in 20 μL PBS in the left ear and with 3 μg Bet v1 in 20 μL PBS in the right ear on Day 25 and 27. Rats were sacrificed by decapitation on days 28 and 29 at which point the microneedle and intradermal injection delivery of allergen experiments was conducted and blood samples was collected. Rats were not fasted before sacrifice; they were already sedated with hypnorrm/midazolam. On the day of sacrifice, the rats were sedated and weighed before their abdomens were shaved. The Transepidermal water loss (TEWL) was measured once for each site of microneedle insertion (measuring for 20 sec) and Evan's blue tinction was injected into the tail vein. Two minutes passed before the microneedle insertion/transdermal injection was performed as shown in FIG. 12B. When inserting at the middle site, 15 minutes passed before sacrificing the animal. TEWL was measured after microneedle insertion, but before sacrificing the animal. Once the animal was sacrificed, skin samples were dissected, stretched and photographed, and blood samples were collected. Skin samples were collected using a biopsy pen after the skin had been stretched to original size and photographed.

### Results

As shown in FIG. 12B, each rat has 8 sites of insertion for the different solutions that are inserted via microneedles or intradermal injections. Each site is lettered from A to H: A, B, C are Bet v1 allergen; D, E, F are birch pollen extract (BPE); G is 48/80 (positive control); H is PBS (negative control). Each solutions' insertion place has been randomized so that there are no biased results i.e. each rat has different insertion places for each solution.

Since the insertion site of each solution is randomized, the order of the solutions are different in FIG. 12C compared with the schematic illustration of FIG. 12B. The black coloration was visible for some samples and said coloration was more visible for the intradermal injections compared with the microneedle insertions (FIG. 12C). The coloration for the microneedle insertion was quantified in FIG. 12D. It is evident that a higher concentration of BPE resulted in an increased allergic reaction in the sensitized rats (right panel) compared with the naive rats (left panel) and a concentration as low as 60 μg/mL was enough to show this difference. The same tendency applies to the Bet v1 antigen, however, the concentration should be at least 600 μg/mL to induce an allergic reaction in the sensitized rats compared with the naive rats.

### Conclusion

Microneedles coated with an allergen, such as BPE or Bet v1, can induce an allergic reaction in sensitized rats and this immunoresponse is quantifiable and increases as the concentration of allergen is increased.

### Example 8—Overall Conclusion

In conclusion, it was possible to manufacture and use a pyrolytic carbon electrode for measurement of histamine in mast cell medium. As demonstrated in example 2, it was possible to coat the electrode with enzyme and use the enzyme to convert histamine, present in mast cell medium, to detect an electrochemical signal, which could be recorded on a monitoring device.

The data recorded could later be used to calculate the histamine concentration within the mast cell medium.

Further, as seen in example 2, a process for manufacture the needles with integrated electrodes was presented and their shape and size was optimized (as seen in example 4).

In addition, examples 5-6 demonstrated that coated microneedles can deliver an antigen to human or pig skin and a tendency for an increased immunoresponse for increasing concentrations of compound 48/80 was observed, indicating a successful delivery of the compound to the rat skin. As seen in example 7, microneedles coated with an allergen, such as BPE or Bet v1, can induce an allergic reaction in sensitized rats and this immunoresponse is quantifiable and increases as the concentration of allergen is increased.

Overall, the examples have shown that microneedles can be used to deliver an antigen to a skin sample and the immunoresponse induced is quantifiable and increases as the concentration of the allergen is increased. In addition, it has been demonstrated that an allergic reaction, exemplified by mast cell degranulation and histamine release can be detected electrochemically. By combining these two observations, it has been demonstrated that the present invention would work.

### REFERENCES

Tehrani et al 2021-Hydrogen Peroxide Detection Using Prussian Blue-Modified 3D Pyrolytic Carbon Microelectrodes-Electroanalysis.
Morales et al 2018-Guide to Selecting a Biorecognition Element for Biosensors-Bioconjugate Chemistry.
Kondzior et al 2020-Antibody-Electroactive Probe Conjugates Based Eelctrochemical Immunosensors-Sensors
Puthongkham et al 2019-Mechanism of Histamine Oxidation and Electropolymerization at Carbon Electrodes-Analytical Chemistry

Although the present invention has been described in connection with the specified embodiments, it should not be construed as being in any way limited to the presented examples.

The scope of the present invention is to be interpreted in the light of the accompanying claim set. In the context of the claims, the terms “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

1-17. (canceled)

18. A system for determining an allergic reaction in skin, the system comprises:
allergen or allergens,
a microneedle array with a microneedle configured to receive said allergen or allergens, for introducing said allergen or allergens into the skin, wherein the microneedle is able to detect an electrochemical signal as a response to the introduced allergen, and
a monitoring device for receiving the electrochemical signal detected by said microneedle, said electrochemical signal being indicative for an allergic reaction in the skin.

19. The system according to claim 18, wherein said electrochemical signal is detected by said microneedle in the skin.

20. The system according to claim 18, wherein said electrochemical signal is detected by said microneedle outside the skin.

21. The system according to claim 18, wherein said electrochemical signal is triggered by biomarkers released by activated mast cells.

22. The system according to claim 18, wherein the electrochemical signal is triggered by biomarkers selected from the list consisting of histamine, chymase, and tryptase.

23. The system according to claim 18, wherein the electrochemical signal is triggered by histamine released by activated mast cells.

24. The system according to claim 21, wherein said microneedle array can detect signals triggered by said biomarkers in concentration below 900 nM.

25. The system according to claim 18, wherein the monitoring device is configured to receive, process and/or record an electrochemical signal from one or more electrode(s) arranged in said microneedle.

26. The system according to claim 18, wherein the allergic reaction can be detected within an hour.

27. The system according to claim 18, wherein the allergic reaction can be detected within 20 minutes.

28. The system according to claim 18, wherein the allergic reaction can be detected within 10 minutes.

29. The system according to claim 18, wherein the system is adapted for measuring said electrochemical signal as a response to the introduced allergen so as to provide a quantitative indication for said allergic reaction in the skin, preferably on a relative scale.

30. A microneedle array comprising:
a microneedle configured to receive an allergen, wherein the microneedle comprises an electrochemical sensor configured to receive, process and/or record an electrochemical signal triggered by the allergen.

31. The microneedle array according to claim 30, wherein the microneedle array comprises a plurality of microneedles arranged in a predetermined pattern.

32. The microneedle array according to claim 30, wherein the microneedle array comprises at least 2 microneedles.

33. The microneedle array according to claim 30, wherein the microneedles are connectable to a monitoring device in groups of 2 or more.

34. The microneedle array according to claim 30, wherein the microneedles are individually connectable to a monitoring device.

35. The microneedle array according to claim 30, wherein the microneedle array comprises a conductive material, preferably the microneedle array comprises pyrolytic carbon.

36. The microneedle array according to claim 30, wherein the of microneedles have a length of at least 400 μm.

37. A set of parts for detecting an allergic reaction in the skin, the set of parts comprising:
allergen or allergens,
a microneedle array according to claim 30
at least one monitoring device for receiving the electrochemical signal detected by said microneedle array in the skin, said electrochemical signal being indicative for an allergic reaction in the skin.
